(19) United States
(12) Patent Application Publication
(10) Pub. No.: US 2022/0395469 A1
(43) Pub. Date: Dec. 15, 2022

(54) TRANSDERMAL DRUG DELIVERY SYSTEM FOR KETAMINE

(71) Applicant: GUANGZHOU DAZHOU BIOMEDICINE LTD., Guangzhou (CN)

(72) Inventors: Huadong TANG, McLean, VA (US); Hock S. TAN, East Brunswick, NJ (US); Michael MAYERSOHN, Marana, AZ (US)

(21) Appl. No.: 17/838,810

(22) Filed: Jun. 13, 2022

Related U.S. Application Data
(63) Continuation of application No. 16/603,729, filed on Oct. 8, 2019, now abandoned.
(60) Provisional application No. 62/487,587, filed on Apr. 20, 2017.
(60) Provisional application No. 62/549,734, filed on Aug. 24, 2017.

(51) Int. Cl. A61K 9/70; A61K 31/135

(52) U.S. Cl. CPC A61K 9/7084; A61K 31/135

(57) ABSTRACT

Provided herein are ketamine gel formulations, transdermal delivery devices comprising ketamine, methods of preparation and methods of using thereof. The transdermal delivery device can be a drug-in-reservoir (DIR) patch comprising ketamine, which typically includes a backing layer, a reservoir layer comprising a ketamine gel formulation, a rate-controlling membrane, an adhesive layer, and a release liner. The ketamine gel formulation generally includes one or more skin permeation enhancers. The transdermal delivery devices can be configured, for example, by adjusting the ketamine gel formulation and other release control mechanism, to provide certain skin flux characteristics, and can be used for treating a variety of indications such as depression and/or pain.

### CROSS REFERENCE TO RELATED APPLICATIONS

This application claims benefit of U.S. Provisional Application No. 62/487,587, filed on Apr. 20, 2017, and 62/549,734, filed on Aug. 24, 2017, the content of each of which is incorporated herein by reference in its entirety.

### BACKGROUND OF THE INVENTION

### Field of the Invention

In various embodiments, the present invention generally relates to ketamine formulations, transdermal delivery device comprising ketamine, methods of preparation thereof, and methods of using thereof.

### Background Art

Major depressive disorder (MDD) is a disabling psychiatric illness. Lifetime prevalence of MDD is approximately 16%. Kessler et al., JAMA, 289(23):3095-105 (2003). There are three primary classes of antidepressants that are commonly prescribed for MDD: (1) monoamine oxidase inhibitors (MAOIs); (2) tricyclics; and (3) serotonin-norepinephrine reuptake inhibitors (SNRIs) and selective serotonin reuptake inhibitors (SSRIs). There are significant limitations with the use of current antidepressants, including limited efficacy, delayed onset of action, and adverse side effects. Antidepressants have been found to be only about 20-30% more effective than placebo. The delay of onset varies from weeks to months, which may result in adverse events, including but not limited to increased vulnerability for suicide, decrease in compliance, and increase in social and economic burden. Common side effects of these antidepressants include nausea, insomnia, anxiety, weight-loss/gain, drowsiness, headache, loss of sex drive, and/or blurred vision. Penn and Tracey. Ther Adv. Psychopharmacol., 2(5):179-188 (2012).

Pain can present as a disabling physical illness. One type of pain, neuropathic pain, is a complex chronic pain state often accompanied by tissue injury. The occurrence of pain with neuropathic characteristics is about 6.9-10% of the general population. Hecke et al., Pain, 155(4):654-62 (2014). Symptoms of neuropathic pain include spontaneous burning, shooting pain, hyperalgesia, and allodynia. Patients with neuropathic pain often have conditions that are associated with other significant health issues, including depression, sleep problems, and loss of independence. Bouhassira et al., Pain, 136(3):380-7 (2008). Neuropathic pain can be caused by a variety of mechanisms, including infection, central or peripheral nerve injury, stroke, multiple sclerosis, diabetes mellitus, sarcoidosis, toxic agents (e.g., alcohol, chemotherapy), inherited or genetic neuropathy, and Complex Regional Pain Syndrome (CRPS). CRPS is an intractable form of pain, often resistant to a variety of conventional therapies. Correll et al., Pain Med., 5(3):263-75 (2004). Neuropathic pain is difficult to treat, with only about 40-60% of patients achieving partial relief. Treatment for neuropathic pain includes antidepressants, anticonvulsants, and/or topical pain management medications. Niesters et al., Expert Opin. Drug Metab. Toxicol., 8(11):1409-17 (2012); Dworkin et al., Pain, 132(3):237-51 (2007).

Ketamine can act as a non-competitive, N-methyl-D-aspartate (NMDA) receptor antagonist, and has been indicated, for example, for treatment as an anesthetic, sedative, and analgesic. For example, ketamine has been demonstrated to be an effective antidepressant, with rapid onset (within about 2 hours of administration) and sustained antidepressant effect (from days to, in some cases, a week or two after administration). Berman et al., Biol. Psychiatry, 47(4):351-54 (2000). The NMDA receptor pathway also plays an important role in pain, including for example neuropathic pain. Animal studies and human clinical studies have shown the efficacy of ketamine in the treatment of chronic neuropathic pain. Correll et al., Pain Med. 5(3):263-75 (2004); Sigtermans et al., Pain, 145(3):304-11 (2009).

Despite the wide range of possible indications, existing ketamine formulations and/or methods of treatments have various drawbacks. For example, IV administration of ketamine presents numerous challenges. First, the patient incurs increased costs to receive IV administration. Second, IV administration is inconvenient for the patient, and may lead to reduced compliance. Third, the rapid initial rise in ketamine plasma concentrations following IV administration to the maximum plasma concentration (Cₘₐₓ) can cause adverse side effects, including drug toxicity, psychotomimetic problems, and increased potential for addiction. Moreover, because ketamine has a short half-life (about 2 hours), this immediate release delivery of ketamine by IV administration may result in little to no ketamine remaining in plasma after about 4 hours, necessitating frequent and repeated dosing to maintain therapeutic plasma levels. Fourth, without additional safeguards. IV administration of ketamine may be susceptible to abuse.

An intranasal formulation of the S-enantiomer of ketamine, esketamine, is under development and in clinical study by Janssen. US 2013/0236573 A1, Singh et al., Esketamine For The Treatment of Treatment-Refractory Or Treatment-Resistant Depression. However, intranasal delivery of ketamine presents numerous challenges. It suffers from many of the same immediate release issues faced by IV administration of ketamine: fast time to maximum concentration (Tₘₐₓ), high Cₘₐₓ, increased risk of side effects like drug toxicity, and the need for frequent, multiple dosing to maintain therapeutic plasma concentrations. Frequent administration of intranasal ketamine may increase the risk of irritating and damaging the nasal epithelium, which in turn may reduce patient compliance. Also, intranasal administration is associated with high variability in absorption among subjects. Kublik et al., Adv. Drug Deliv. Rev. 29:157-77 (1998). Further, the rapid rise in ketamine plasma concentration following intranasal administration may cause adverse side effects, such as drug toxicity. Moreover, intranasal delivery of ketamine, without additional safeguards, is highly susceptible to abuse. Other routes of administration of ketamine, including parenteral administration of ketamine (e.g., subcutaneous, intramuscular, etc.) suffer from many of these same challenges.

While oral administration (i.e., tablet or capsule) is typically a convenient route for the patient, the metabolic and pharmacokinetic properties of ketamine make oral administration less suitable. Ketamine has a high systemic (primarily hepatic) clearance of about 19 ml/min·kg, a rate which approaches liver plasma flow. Thus, ketamine is subject to substantial pre-systemic metabolism, or first-pass effect, in the liver and gut wall by metabolic enzymes, such as cytochrome P450 enzymes (CYP450). Consequently, the absolute oral bioavailability of ketamine in humans is only about 10-20%. Due to this first-pass effect, there is an increased risk for drug-drug interactions (DDI) with drugs that can inhibit or induce CYP450s. Clements et al., J Pharm Sci, 71(5):539-42 (1981); Fanta, et al., Eur. J. Clin. Pharmacol., 71:441-47 (2015); Peltoniemi et al., Basic & Clinical Pharmacology & Toxicology, 111:325-332 (2012). Moreover, ketamine tablets or capsules are easily abused.

In light of the above, novel ketamine formulations and/or methods of using thereof are still needed.

### BRIEF SUMMARY OF THE INVENTION

In various embodiments, the present disclosure relates to transdermal delivery devices comprising ketamine. The transdermal delivery device typically includes at least a backing layer, a reservoir layer comprising ketamine, and an adhesive layer. The ketamine is generally present in an amount of about 2% to about 30% (e.g., about 2%, about 2.5%, about 5%, about 10%, about 15%, about 18%, about 20%, about 25%, about 30%, or any ranges in between the specified values) by weight of the reservoir layer. In some embodiments, the transdermal delivery device is configured, for example, by adjusting the ketamine formulation in the reservoir layer and/or various rate-controlling mechanisms of the transdermal delivery device, to provide skin flux characteristics as described herein. Preferably, the skin flux characteristics are such that the transdermal delivery device, when administered to a subject (e.g., a human subject), can provide (1) sufficiently high and long-lasting exposure of ketamine in the plasma of the subject for the treatment of various indications, such as depression, anxiety, pains, etc.; and/or (2) slow-rising ketamine concentrations of ketamine in the plasma of the subject, which can reduce adverse side effects of ketamine commonly associated with the high Cmax upon administration of ketamine by approaches such as short-term IV infusion, intranasal delivery, etc. These side effects include, but are not limited to, psychomimetic side effects and the more common dissociative symptoms, which are noted to occur around 2 hours and disappear quickly after about 4 hours, so are the hemodynamic changes. The lowered Cmax derived from embodiments of the invention can reduce abuse potential by reduction of the self-rewarding feedback caused by “high” or “dissociative effects” due to high Cmax. The slow-rising pharmacokinetic (“PK”) profile can also reduce potential neurotoxicity, as Olney's paper suggested that the neuro-cytomorphological changes by NMDAR antagonists are mediated by Cmax, rather than total dose (or area under the curve (“AUC”)). See, e.g., Olney, J. W., et al., Science 244:1360-1362, 1989. Prior low dose exposure of NMDAR antagonists could lead to insensitivity of such changes to high exposure (that is, tolerance to neuro-cytotoxicity developed after low exposure).

In some embodiments, the transdermal delivery device is a drug-in-reservoir (DIR) patch comprising ketamine, which typically includes a backing layer, a reservoir layer comprising, consisting essentially of, or consisting of a ketamine gel formulation, a rate-controlling membrane, an adhesive layer, and a release liner. In a typical design, the reservoir layer is sandwiched between the backing layer and the rate-controlling membrane. The backing layer is typically an impermeable film. The adhesive layer is generally configured to contact the skin of a subject and the adhesive surface is generally protected by the release liner. Preferably, the DIR patches described herein are storage stable and are suitable for application to the skin of a human subject, e.g., with minimal skin irritation.

In some embodiments, the ketamine gel formulation can include ketamine, a solvent, one or more permeation enhancers, and a gel-forming agent. In some embodiments, ketamine can be present in the gel formulation in an amount of about 2% to about 30% (e.g., about 2%, about 2.5%, about 5%, about 10%, about 15%, about 18%, about 20%, about 25%, about 30%, or any ranges in between the specified values) by weight of the gel formulation. The solvent is typically present in an amount of about 40% to about 75% by weight of the gel formulation. In some embodiments, the solvent can comprise ethanol, water, propylene glycol, acetone, isopropyl alcohol, butylene glycol, dimethyl sulfoxide (DMSO), dimethyl acetamide (DMA), or a combination thereof. The permeation enhancer is typically present in an amount of about 5% to about 25% by weight of the gel formulation. In some embodiments, the permeation enhancer can include one or more compounds chosen from sulfoxides, alcohols, alkanols, esters, glycols, and surfactants. For example, in some embodiments, the permeation enhancer can include one or more compounds chosen from dimethyl sulfoxide (DMSO), oleic alcohol, oleic acid, oleyl oleate, levulinic acid, propylene glycol, dipropylene glycol, ethanol, and surfactants such as Tween 80. Various gel-forming agents are suitable. In some embodiments, the gel-forming agent can comprise a hydroxypropylcellulose (HPC), hydroxypropyl methyl cellulose (HPMC), polyvinyl pyrrolidone (PVP such as BASF's Kollidon), polyacrylic acid (such as Carbopol), sodium CMC (carboxyl methyl cellulose), or a combination thereof. The gel-forming agent is generally present in a gel-forming amount. In some embodiments, the gel-forming agent is included such that the final ketamine gel formulation has a viscosity of about 15,000 cP to about 45,000 cP.

The adhesive layer generally includes a pressure sensitive adhesive, for example, a polyisobutylene (PIB) adhesive, a silicone polymer adhesive, an acrylate copolymer adhesive, or a combination thereof. In some embodiments, the adhesive layer is about 1.5 mils to about 10 mils (e.g., about 1.5 mils to about 2 mils) thick.

The rate-controlling membrane is typically a microporous membrane. In some embodiments, the microporous membrane can include a polypropylene film, a poly ethylene vinyl acetate (EVA) film, or a combination thereof. The rate-controlling membrane is typically in between the reservoir layer and the adhesive layer.

The amount of ketamine in the transdermal delivery device can be adjusted according to its applications. For example, in some embodiments, the reservoir layer comprises an amount of ketamine sufficient to provide about 0.1 mg/day/cm² to about 30 mg/day/cm² (preferably, about 1 mg/day/cm² to about 5 mg/day/cm² or about 2 mg/day/cm² to about 10 mg/day/cm² of ketamine) of ketamine over a period of time selected from about 8 hours, about 12 hours, about 18 hours, about 24 hours, about 2 days, about 3 days, about 4 days, about 5 days, about 6 days, and about 7 days.

The transdermal delivery device can have various sizes. For example, in some embodiments, the transdermal delivery device has an active surface area of about 5 cm² to about 300 cm², for example, about 10 cm² to about 100 cm².

In some embodiments, the transdermal delivery device also includes an abuse deterrent mechanism. For example, in some embodiments, the transdermal delivery device comprises an abuse deterrent agent selected from aversive agents, such as capsaicin, apomorphine, denatonium, sodium laurel sulfate, niacin and combinations thereof. The abuse deterrent agent can be present in the reservoir layer, the adhesive layer, and/or in a separate layer.

Certain embodiments of the present disclosure are directed to methods of administering ketamine to a subject in need thereof, e.g., for antagonizing NMDA receptors, treating diseases or disorders where antagonizing NMDA receptor is beneficial, or treating depression, anxiety, and/or pain. In some embodiments, the method comprises applying a transdermal ketamine formulation (e.g., a transdermal delivery device described herein) to the subject. In some embodiments, applying the transdermal ketamine formulation provides a therapeutically effective concentration of ketamine for an extended period, yet provides a lower Cₘₐₓ compared to that of a dose-equivalent intravenous or intranasal formulation. In some embodiments, the subject is a human subject characterized as having depression, anxiety, and/or pain. In some embodiments, the transdermal ketamine formulation (e.g., a transdermal delivery device described herein) is applied to provide a pharmacokinetic profile in the human subject characterized by (1) an initial slow-rising phase; and/or (2) a sustained release phase with ketamine concentrations in the plasma being substantially constant (e.g., with fluctuation within about 0.5 to about 2 fold relative to the mean value).

In some embodiments, additional active agent(s) can be incorporated in the ketamine gel formulation or transdermal delivery device described herein, or otherwise be administered concurrently or sequentially with the ketamine gel formulation or transdermal delivery device described herein, for example, to counteract adverse effects, and/or to enhance the antidepressant or pain management effect of ketamine. Examples for enhancing antidepressant effect include, but are not limited to, antagonists of group II metabotropic glutamate receptors, such as LY341495, Podkowa et al., Psychopharmacology (Berl) 233 (15-16), 2901-2914 (Jun. 11, 2016). Examples for reducing side effects with ketamine, especially psychotomimetic and sympathomimetic, include, but are not limited to, alpha-2 agonists such as clonidine. Lenze, World J Biol Psychiatry, 17(3):230-8 (2016).

### BRIEF DESCRIPTION OF THE FIGURES

FIG. 1 shows a typical drug in reservoir (DIR) transdermal patch design. In general, the liquid (gel) reservoir design includes a drug-loaded reservoir sandwiched between a backing film and a rate-controlling membrane, and a skin-contacting pressure sensitive adhesive (PSA) layer (FIG. 1). The liquid reservoir compartment contains the drug and one or more skin permeation enhancers. Typically, the system is supported by an impermeable backing film, and the adhesive surface is protected by a release liner.

FIG. 2 shows a typical process flow for preparing a DIR transdermal patch.

FIG. 3 presents plots of skin flux data for three patch formulations of ketamine. A drug-in-adhesive (DIA) patch (Patch 5) with ketamine concentration of 15% achieved a cumulative flux of 0.73 mg/cm² at 24 hours. In comparison, the cumulative flux at 24 hours for the DIR patch (Patch 7) with ketamine concentration of 15% was found to be 10 fold higher, at 7.78 mg/cm². And the cumulative flux at 24 hours for the DIR patch (Patch 6) with ketamine concentration of 10% was found to be 5.17 mg/cm², also considerably higher than that observed for the DIA patch.

FIGS. 4A-4E show estimated time-course concentration profiles of ketamine from 15% ketamine DIR patches with varying patch sizes of 5 cm², 50 cm², and 200 cm², designed for 72-hour, 48-hour, 24-hour, 18-hour, and 12-hour delivery, respectively. The DIR patches include the same gel formulation and/or have the same skin flux characteristics as those of patch 7, if configured as (or scaled to) a 72-hour delivery patch.

FIG. 4F shows estimated time-course concentration profiles of ketamine from Patches 5 and 6 with a patch size of 200 cm.

FIG. 5 presents plots of skin flux data showing stability and repeatability of patches containing 15 wt % ketamine.

### DETAILED DESCRIPTION OF THE INVENTION

In various embodiments, the present disclosure relates to transdermal delivery devices comprising ketamine. The transdermal delivery device can be advantageous in many aspects over conventional intravenous infusion or intranasal ketamine delivery. For example, administering ketamine with the transdermal delivery device herein can reduce the risk of side effects and can improve patient compliance. The current widely used dosage form, 40-min intravenous infusion at 0.5 mg/kg, is not a viable commercial product that can be widely, conveniently and cost-effectively prescribed, as it has many critical issues, such as side effects, abuse potential, high cost and inconvenience. The major side effects of the 40-min IV infusion at 0.5 mg/kg include: psychomimetic effects, dissociative symptoms, elevated blood pressure and/or heart rate (Murrough et al., Am J Psychiatry. 2013 October; 170(10):1134-42). Recently, FDA emphasized publicly on the potential neurotoxicity from ketamine, which was observed in rats (Olney. J. W., et al., Science 244:1360-1362, 1989; Annual Meeting of American Society of Clinical Psychopharmacology, May 29-Jun. 2, 2017). These side effects from the IV dosing were also observed with intranasal administration of ketamine, and such intranasal administration has to be implemented in medical environment, such as in hospitals/clinics with physicians/staff monitoring, thus adding a significant cost to the patients and healthcare.

The transdermal delivery system of embodiments of this invention can, at least partially, overcome or minimize these issues by providing a slow rising pharmacokinetic profile with prolonged exposure and a substantially lowered Cmax (comparing to 40-min infusion or intranasal administration at the same doses). The primary treatment mode involves administration of ketamine via constant rate IV infusion, generally given as a 40-minute infusion of 0.5 mg ketamine/kg. The psychomimetic side effects and the more common dissociative symptoms are noted to occur around 2 hours and disappear quickly after about 4 hours, so are the hemodynamic changes. Ketamine has a short T½ of about 2 hours. Therefore, ketamine plasma concentrations are very low at or after 4 hours post-infusion. This correlation of drug concentrations and side effects strongly suggested that these side effects are driven by Cmax. A slow rising pharmacokinetic profile with prolonged exposure and a substantially lower Cmax of ketamine can therefore significantly reduce risks of these Cmax driven side effects.

Further, the transdermal delivery device herein can reduce abuse potential. A patch itself is a device that may reduce the abuse potential. The common abuse approaches of ketamine include oral assumption, snorting, and IV/IM injection (U.S. Department of Justice http://www.justice.gov/archive/ndic/pubs4/4769/). To abuse the ketamine in patches, one has to be able to extract the ketamine, which is a significant barrier. Abuse deterrent formulations (ADF) can also be incorporated into the transdermal delivery device herein. Lowered Cmax with prolonged exposure reduces the dissociative effects, or the “high” feeling, resulting from the high concentration of ketamine, reduces a self-rewarding feedback and thus abuse potential.

Also, the transdermal delivery device herein can reduce potential neurotoxicity with a slow rising pharmacokinetic profile: Olney's paper suggested that the neuro-cytomorphological changes by NMDAR antagonists are mediated by Cmax, rather than total dose or AUC. Prior low dose exposure of NMDAR antagonists could lead to insensitivity of such changes to high exposure (that is, tolerance to neuro-cytotoxicity developed after low exposure). Therefore, the slow-rising concentration can be advantageous by offering insensitivity to neuro-cytotoxicity with low exposure at early times.

In some embodiments, the transdermal delivery device described herein can also be characterized by a high ketamine permeability profile. For example, in some embodiments, the DIR patches described herein can provide higher transdermal permeation rates than those observed from the DIA patches with similar drug concentrations. Thus, the DIR patches can provide higher systemic ketamine exposures in humans and allow delivery of sufficiently high and long-lasting exposure of ketamine for the treatment of different indications, such as depression, anxiety, pains, etc.

Transdermal Delivery Device Comprising Ketamine

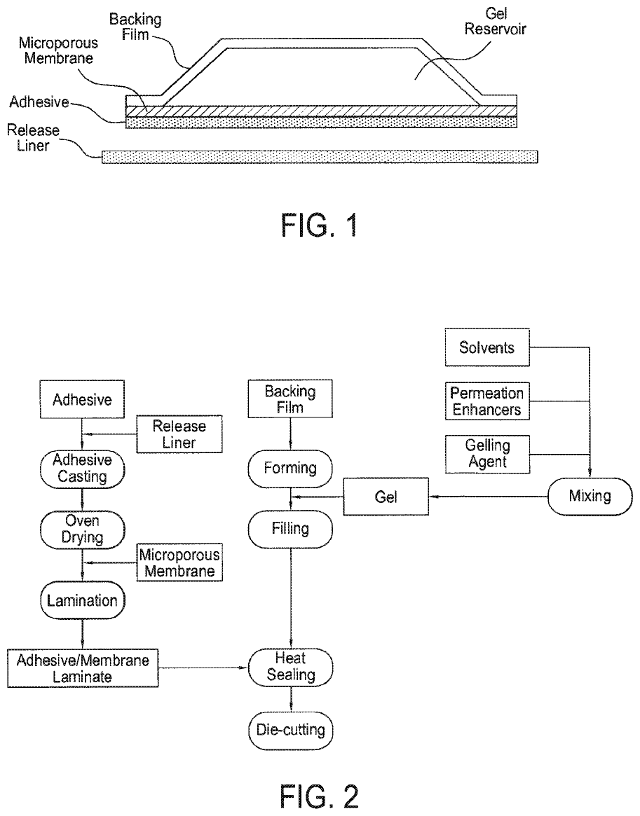

Certain embodiments of the present disclosure are directed to a transdermal delivery device for administering ketamine. In some embodiments, the transdermal delivery device comprises a backing layer; a reservoir layer comprising ketamine in an amount of about 2% to about 30% by weight of the reservoir layer; and an adhesive layer defining an active surface area. The reservoir layer and the adhesive layer are typically two separate layers that are laminated to each other or separated, for example, by a rate-controlling membrane. In preferred embodiments, the transdermal delivery device is a drug-in-reservoir (DIR) patch, e.g., a gel reservoir-type transdermal patch. In general, the liquid (gel) reservoir design includes a drug-loaded reservoir layer sandwiched between a backing film and a rate-controlling membrane such as a microporous membrane, and a skin-contacting pressure sensitive adhesive (PSA) layer (see, e.g., FIG. 1). The liquid reservoir compartment can contain the drug and one or more skin permeation enhancers. Typically, the DIR patch is supported by an impermeable backing film, and the adhesive surface is protected by a release liner.

Ketamine can be present in the reservoir layer of the transdermal delivery device in various amounts. In some embodiments, the reservoir layer comprises ketamine in an amount of about 2%, about 2.5%, about 5%, about 10%, about 15%, about 18%, about 20%, about 25%, about 30%, or any ranges in between the recited values, by weight of the reservoir layer. In some specific embodiments, the reservoir layer comprises ketamine in the amount of about 2%, about 2.5%, about 5%, about 10%, about 15%, about 18% by weight of the reservoir layer. Typically, the reservoir layer contains substantially all of the ketamine of the transdermal delivery device. However, the adhesive layer can also include ketamine, for example, a priming dose or through equilibration with the reservoir layer.

It was proposed that the anesthetic and/or antidepressant effect of ketamine is mainly through the action of S-ketamine because in vitro S-ketamine has about a 4-fold greater affinity than the R-ketamine on NMDA receptor binding. However, animal model studies have suggested that R-ketamine is more effective as an antidepressant than S-ketamine. In addition, R-ketamine was shown to be free of psychotomimetic side effects and abuse liability. Yang et al., Transl. Psychiatry, 5(e632):1-11 (2015). The ketamine in the transdermal delivery device described herein is not limited to a particular enantiomer and can be in a racemic form, a substantially pure S-enantiomer (e.g., with less than 10% R-isomer, less than 5% R-isomer, less than 1% R-isomer, or less than 0.1% R-isomer), a substantially pure R-enantiomer (e.g., with less than 10% S-isomer, less than 5% S-isomer, less than 1% S-isomer, or less than 0.1% S-isomer), or a mixture of S- and R-isomers in any ratio.

Skin Flux Characteristics

The transdermal delivery device described herein is preferably configured to provide certain desired skin flux characteristics. For example, in some embodiments, the transdermal delivery device is configured to provide one or more of the following skin flux characteristics when tested in vitro using human cadaver skin: (a) a cumulative ketamine permeated of about 0.04 mg/cm² to about 3 mg/cm² at 12 hours post administration based on the active surface area; (b) a cumulative ketamine permeated of about 0.8 mg/cm² to about 20 mg/cm² at 24 hours post administration based on the active surface area; (c) a cumulative ketamine permeated of about 2.5 mg/cm² to about 65 mg/cm² at 48 hours post administration based on the active surface area; (d) a cumulative ketamine permeated of about 3 mg/cm² to about 85 mg/cm² at 72 hours post administration based on the active surface area; (e) an average flux of ketamine of about 0.005 mg/cm²*h to about 0.4 mg/cm²*h from 4 hours to 12 hours post administration; (f) an average flux of ketamine of about 0.06 mg/cm²*h to about 1.4 mg/cm²*h from 12 hours to 18 hours post administration; (g) an average flux of ketamine of about 0.06 mg/cm²*h to about 1.4 mg/cm²*h from 12 hours to 24 hours post administration; (b) a steady state flux of ketamine of about 0.06 mg/cm²*h to about 1.8 mg/cm²*h; (i) an average flux of ketamine of about 0.08 mg/cm²*h to about 1.8 mg/cm²*h from 24 hours to 48 hours post administration; and (j) an average flux of ketamine of about 0.03 mg/cm²*h to about 0.9 mg/cm²*h from 48 hours to 72 hours post administration.

Both cumulative ketamine permeated and ketamine permeation rate are important flux characteristics. In some preferred embodiments, the transdermal delivery device is configured to provide one or more (e.g., 1, 2, 3 or 4) of skin flux characteristics related to cumulative ketamine permeated, (a) to (d), and one or more (e.g., 1, 2, 3, 4, 5 or 6) of skin flux characteristics related to ketamine permeation rate, such as average flux and steady state flux, (e) to (j), when tested in vitro using human cadaver skin. For example, in some embodiments, the transdermal delivery device is designed for delivery of ketamine for about 24 hours or more, and the skin flux characteristics can include at least (a), (b), (e), (f), and (g). In some embodiments, the transdermal delivery device is designed for delivery of ketamine for about 48 hours or more, and the skin flux characteristics can include at least (a), (b), (c), (e), (f), (g), (h), and (i). In some embodiments, the transdermal delivery device is designed for delivery of ketamine for about 48 or 72 hours or more, and the skin flux characteristics can include at least (c), (h), and (i). In some embodiments, the transdermal delivery device is designed for delivery of ketamine for about 72 hours, and the skin flux characteristics can include all of (a)-(j).

Preferred flux characteristics can vary according to the different applications of the transdermal delivery devices described herein. In some embodiments, the ketamine is present in the reservoir layer in an amount of about 2% to about 30%, preferably, about 2% to about 20% (e.g., about 2% to about 10%, about 2% to about 5%, etc.), more preferably, about 5% to about 15% by weight of the reservoir layer, and the transdermal delivery device can be configured to provide one or more of the following skin flux characteristics when tested in vitro using human cadaver skin: 1) a cumulative ketamine permeated of about 0.04 mg/cm² to about 0.2 mg/cm² at 12 hours post administration based on the active surface area; 2) a cumulative ketamine permeated of about 0.8 mg/cm² to about 3.5 mg/cm² at 24 hours post administration based on the active surface area; 3) a cumulative ketamine permeated of about 2.5 mg/cm² to about 11 mg/cm² at 48 hours post administration based on the active surface area; 4) a cumulative ketamine permeated of about 3 mg/cm² to about 15 mg/cm² at 72 hours post administration based on the active surface area; 5) an average flux of ketamine of about 0.005 mg/cm²*h to about 0.03 mg/cm²*h from 4 hours to 12 hours post administration; 6) an average flux of ketamine of about 0.06 mg/cm²*h to about 0.26 mg/cm²*h from 12 hours to 18 hours post administration; 7) an average flux of ketamine of about 0.06 mg/cm²*h to about 0.26 mg/cm²*h from 12 hours to 24 hours post administration; 8) a steady state flux of ketamine of about 0.06 mg/cm²*h to about 0.31 mg/cm²*h; 9) an average flux of ketamine of about 0.07 mg/cm²*h to about 0.31 mg/cm²*h from 24 hours to 48 hours post administration; and 10) an average flux of ketamine of about 0.03 mg/cm²*h to about 0.12 mg/cm²*h from 48 hours to 72 hours post administration. In some embodiments, the transdermal delivery device is configured to provide one or more (e.g., 1, 2, 3 or 4) of skin flux characteristics of 1) to 4) and one or more (e.g., 1, 2, 3, 4, 5 or 6) of skin flux characteristics of 5) to 10), when tested in vitro using human cadaver skin. For example, in some embodiments, the transdermal delivery device is designed for delivery of ketamine for about 24 hours or more, and the skin flux characteristics can include at least 1), 2), 5), 6), and 7). In some embodiments, the transdermal delivery device is designed for delivery of ketamine for about 48 hours or more, and the skin flux characteristics can include at least 1) to 3) and 5) to 9). In some embodiments, the transdermal delivery device is designed for delivery of ketamine for about 48 or 72 hours or more, and the skin flux characteristics can include at least 3), 8), and 9). In some embodiments, the transdermal delivery device is designed for delivery of ketamine for about 72 hours, and the skin flux characteristics can include all of 1) to 10).

In some embodiments, the ketamine is present in the reservoir layer in an amount of about 5% to about 30%, preferably, about 5% to about 15%, more preferably, about 5% to about 10% (e.g., about 5%, about 6%, about 7%, about 8%, about 9%, or about 10%), by weight of the reservoir layer, and the transdermal delivery device can be configured to provide one or more of the following skin flux characteristics when tested in vitro using human cadaver skin: 1) a cumulative ketamine permeated of about 0.1 mg/cm² to about 0.4 mg/cm² at 12 hours post administration based on the active surface area; 2) a cumulative ketamine permeated of about 2 mg/cm² to about 7 mg/cm² at 24 hours post administration based on the active surface area; 3) a cumulative ketamine permeated of about 6 mg/cm² to about 25 mg/cm² at 48 hours post administration based on the active surface area; 4) a cumulative ketamine permeated of about 8 mg/cm² to about 30 mg/cm² at 72 hours post administration based on the active surface area; 5) an average flux of ketamine of about 0.013 mg/cm²*h to about 0.05 mg/cm²*h from 4 hours to 12 hours post administration; 6) an average flux of ketamine of about 0.15 mg/cm²*h to about 0.6 mg/cm²*h from 12 hours to 18 hours post administration; 7) an average flux of ketamine of about 0.15 mg/cm²*h to about 0.6 mg/cm²*h from 12 hours to 24 hours post administration; 8) a steady state flux of ketamine of about 0.15 mg/cm²*h to about 0.7 mg/cm²*h; 9) an average flux of ketamine of about 0.19 mg/cm²*h to about 0.7 mg/cm²*h from 24 hours to 48 hours post administration; and 10) an average flux of ketamine of about 0.07 mg/cm²*h to about 0.3 mg/cm²*h from 48 hours to 72 hours post administration. In some embodiments, the transdermal delivery device is configured to provide one or more (e.g., 1, 2, 3 or 4) of skin flux characteristics of 1) to 4) and one or more (e.g., 1, 2, 3, 4, 5 or 6) of skin flux characteristics of 5) to 10), when tested in vitro using human cadaver skin. In some embodiments, the transdermal delivery device can be configured to provide skin flux characteristics (i) at least 1), 2), 5), 6), and 7); (ii) at least 1), 2), and (8), or (iii) at least 1), 2) and 5) to 8), when tested in vitro using human cadaver skin. In some embodiments, the skin flux characteristics can further optionally include (iv) 3) and/or (9); (v) 4) and/or 10); or (vi) a combination of (iv) and (v), when tested in vitro using human cadaver skin. In some embodiments, the transdermal delivery device is designed for delivery of ketamine for about 24 hours or more, and the skin flux characteristics can include at least 1), 2), 5), 6), and 7). In some embodiments, the transdermal delivery device is designed for delivery of ketamine for about 48 hours or more, and the skin flux characteristics can include at least 1) to 3) and 5) to 9). In some embodiments, the transdermal delivery device is designed for delivery of ketamine for about 48 or 72 hours or more, and the skin flux characteristics can include at least 3), 8), and 9). In some embodiments, the transdermal delivery device is designed for delivery of ketamine for about 72 hours, and the skin flux characteristics can include all of 1) to 10).

In some embodiments, the ketamine is present in the reservoir layer in an amount of about 5% to about 30%, preferably, about 5% to about 20% (e.g., about 8%, about 10%, about 12%, about 15%, about 18%, or any ranges between the specified values), more preferably, about 10% to about 15%, by weight of the reservoir layer, and the transdermal delivery device can be configured to provide one or more of the following skin flux characteristics when tested in vitro using human cadaver skin: 1) a cumulative ketamine permeated of about 0.2 mg/cm² to about 1.5 mg/cm² at 12 hours post administration based on the active surface area; 2) a cumulative ketamine permeated of about 4 mg/cm² to about 10 mg/cm² at 24 hours post administration based on the active surface area; 3) a cumulative ketamine permeated of about 13 mg/cm² to about 35 mg/cm² at 48 hours post administration based on the active surface area; 4) a cumulative ketamine permeated of about 17 mg/cm² to about 45 mg/cm² at 72 hours post administration based on the active surface area; 5) an average flux of ketamine of about 0.02 mg/cm²*h to about 0.2 mg/cm²*h from 4 hours to 12 hours post administration; 6) an average flux of ketamine of about 0.3 mg/cm²*h to about 0.7 mg/cm²*h from 12 hours to 18 hours post administration; 7) an average flux of ketamine of about 0.3 mg/cm²*h to about 0.7 mg/cm²*h from 12 hours to 24 hours post administration; 8) a steady state flux of ketamine of about 0.3 mg/cm²*h to about 0.9 mg/cm²*h; 9) an average flux of ketamine of about 0.35 mg/cm²*h to about 0.9 mg/cm²*h from 24 hours to 48 hours post administration; and 10) an average flux of ketamine of about 0.15 mg/cm²*h to about 0.45 mg/cm²*h from 48 hours to 72 hours post administration. In some embodiments, the transdermal delivery device is configured to provide one or more (e.g., 1, 2, 3 or 4) of skin flux characteristics of 1) to 4) and one or more (e.g., 1, 2, 3, 4, 5 or 6) of skin flux characteristics of 5) to 10), when tested in vitro using human cadaver skin. In some embodiments, the transdermal delivery device can be configured to provide skin flux characteristics (i) at least 1), 2), 5), 6), and 7); (ii) at least 1), 2), and (8), or (iii) at least 1), 2) and 5) to 8), when tested in vitro using human cadaver skin. In some embodiments, the skin flux characteristics can further optionally include (iv) 3) and/or (9); (v) 4) and/or 10); or (vi) a combination of (iv) and (v), when tested in vitro using human cadaver skin. In some embodiments, the transdermal delivery device is designed for delivery of ketamine for about 24 hours or more, and the skin flux characteristics can include at least 1), 2), 5), 6), and 7). In some embodiments, the transdermal delivery device is designed for delivery of ketamine for about 48 hours or more, and the skin flux characteristics can include at least 1) to 3) and 5) to 9). In some embodiments, the transdermal delivery device is designed for delivery of ketamine for about 48 or 72 hours or more, and the skin flux characteristics can include at least 3), 8), and 9). In some embodiments, the transdermal delivery device is designed for delivery of ketamine for about 72 hours, and the skin flux characteristics can include all of 1) to 10).

In some embodiments, the ketamine is present in the reservoir layer in an amount of about 5% to about 30%, preferably, about 5% to about 15%, about 5% to about 10% (e.g., about 5%, about 10%), or about 10% to about 20%, (e.g., about 10%, about 15%, about 18%, or about 20%), by weight of the reservoir layer, and the transdermal delivery device can be configured to provide one or more of the following skin flux characteristics when tested in vitro using human cadaver skin: 1) a cumulative ketamine permeated of about 0.2 ng/cm² to about 0.4 mg/cm² at 12 hours post administration based on the active surface area; 2) a cumulative ketamine permeated of about 4 mg/cm² to about 7 mg/cm² at 24 hours post administration based on the active surface area; 3) a cumulative ketamine permeated of about 13 mg/cm² to about 25 mg/cm² at 48 hours post administration based on the active surface area; 4) a cumulative ketamine permeated of about 17 mg/cm² to about 30 mg/cm² at 72 hours post administration based on the active surface area; 5) an average flux of ketamine of about 0.02 mg/cm²*h to about 0.05 mg/cm²*h from 4 hours to 12 hours post administration; 6) an average flux of ketamine of about 0.3 mg/cm²*h to about 0.6 mg/cm²*h from 12 hours to 18 hours post administration; 7) an average flux of ketamine of about 0.3 mg/cm²*h to about 0.6 mg/cm²*h from 12 hours to 24 hours post administration; 8) a steady state flux of ketamine of about 0.3 mg/cm²*h to about 0.7 mg/cm²*h; 9) an average flux of ketamine of about 0.35 mg/cm²*h to about 0.7 mg/cm²*h from 24 hours to 48 hours post administration; and 10) an average flux of ketamine of about 0.15 mg/cm²*h to about 0.3 mg/cm²*h from 48 hours to 72 hours post administration. In some embodiments, the transdermal delivery device is configured to provide one or more (e.g., 1, 2, 3 or 4) of skin flux characteristics of 1) to 4) and one or more (e.g., 1, 2, 3, 4, 5 or 6) of skin flux characteristics of 5) to 10), when tested in vitro using human cadaver skin. In some embodiments, the transdermal delivery device can be configured to provide skin flux characteristics (i) at least 1), 2), 5), 6), and 7); (ii) at least 1), 2), and (8), or (iii) at least 1), 2) and 5) to 8), when tested in vitro using human cadaver skin. In some embodiments, the skin flux characteristics can further optionally include (iv) 3) and/or (9); (v) 4) and/or 10); or (vi) a combination of (iv) and (v), when tested in vitro using human cadaver skin. In some embodiments, the transdermal delivery device is designed for delivery of ketamine for about 24 hours or more, and the skin flux characteristics can include at least 1), 2), 5), 6), and 7). In some embodiments, the transdermal delivery device is designed for delivery of ketamine for about 48 hours or more, and the skin flux characteristics can include at least 1) to 3) and 5) to 9). In some embodiments, the transdermal delivery device is designed for delivery of ketamine for about 48 or 72 hours or more, and the skin flux characteristics can include at least 3), 8), and 9). In some embodiments, the transdermal delivery device is designed for delivery of ketamine for about 72 hours, and the skin flux characteristics can include all of 1) to 10).

In some embodiments, the ketamine is present in the reservoir layer in an amount of about 10% to about 30% (e.g., about 10%, about 15%, about 18%, or about 20%), preferably, about 10% to about 15%, about 10% to about 20%, or about 15% to about 20%, by weight of the reservoir layer, and the transdermal delivery device can be configured to provide one or more of the following skin flux characteristics when tested in vitro using human cadaver skin: 1) a cumulative ketamine permeated of about 0.9 mg/cm² to about 1.5 mg/cm² at 12 hours post administration based on the active surface area; 2) a cumulative ketamine permeated of about 6 mg/cm² to about 10 mg/cm² at 24 hours post administration based on the active surface area; 3) a cumulative ketamine permeated of about 19 mg/cm² to about 35 mg/cm² at 48 hours post administration based on the active surface area; 4) a cumulative ketamine permeated of about 25 mg/cm² to about 45 mg/cm² at 72 hours post administration based on the active surface area; 5) an average flux of ketamine of about 0.1 mg/cm²*h to about 0.2 mg/cm²*h from 4 hours to 12 hours post administration; 6) an average flux of ketamine of about 0.4 mg/cm²*h to about 0.7 mg/cm²*h from 12 hours to 18 hours post administration; 7) an average flux of ketamine of about 0.4 mg/cm²*h to about 0.7 mg/cm²*h from 12 hours to 24 hours post administration; 8) a steady state flux of ketamine of about 0.4 mg/cm²*h to about 0.9 mg/cm²*h; 9) an average flux of ketamine of about 0.5 mg/cm²*h to about 0.9 mg/cm²*h from 24 hours to 48 hours post administration; and 10) an average flux of ketamine of about 0.25 mg/cm²*h to about 0.45 mg/cm²*h from 48 hours to 72 hours post administration. In some embodiments, the transdermal delivery device is configured to provide one or more (e.g., 1, 2, 3 or 4) of skin flux characteristics of 1) to 4) and one or more (e.g., 1, 2, 3, 4, 5 or 6) of skin flux characteristics of 5) to 10), when tested in vitro using human cadaver skin. In some embodiments, the transdermal delivery device can be configured to provide skin flux characteristics (i) at least 1), 2), 5), 6), and 7); (ii) at least 1), 2), and (8), or (iii) at least 1), 2) and 5) to 8), when tested in vitro using human cadaver skin. In some embodiments, the skin flux characteristics can further optionally include (iv) 3) and/or (9); (v) 4) and/or 10); or (vi) a combination of (iv) and (v), when tested in vitro using human cadaver skin. In some embodiments, the transdermal delivery device is designed for delivery of ketamine for about 24 hours or more, and the skin flux characteristics can include at least 1), 2), 5), 6), and 7). In some embodiments, the transdermal delivery device is designed for delivery of ketamine for about 48 hours or more, and the skin flux characteristics can include at least 1) to 3) and 5) to 9). In some embodiments, the transdermal delivery device is designed for delivery of ketamine for about 48 or 72 hours or more, and the skin flux characteristics can include at least 3), 8), and 9). In some embodiments, the transdermal delivery device is designed for delivery of ketamine for about 72 hours, and the skin flux characteristics can include all of 1) to 10).

Transdermal delivery devices with the above flux characteristics can be prepared by those skilled in the art in view of the present disclosure. As described in detail herein, the cumulative ketamine permeated and the average flux can be adjusted, for example, by the reservoir composition (e.g., drug concentration, permeation enhancer, coat weight, etc.) and various release control mechanisms. The time required to achieve a steady state flux of ketamine of the transdermal delivery device can vary and can be adjusted. In some embodiments, the steady state flux of ketamine described above is achieved from about 8 hours, about 12 hours, about 14 hours, about 16 hours, about 18 hours, about 20 hours, about 24 hours, or any ranges between the recited value, post administration. In some preferred embodiments, the steady state flux of ketamine is achieved from about 18 hours to about 24 hours post administration.

Ketamine Gel Formulation

Typically, a ketamine gel formulation comprising ketamine, a solvent, a permeation enhancer, and a gel-forming agent is included in the reservoir layer of the transdermal delivery device described herein. In some embodiments, the ketamine gel formulation is adjusted such that the transdermal delivery device achieves the skin flux characteristics described hereinabove. The ketamine gel formulation herein is also a novel formulation independent of the transdermal delivery device and/or the above skin flux characteristics. Thus, some embodiments of the present disclosure are also directed to the ketamine gel formulation.

The ketamine can be present in the gel formulation in various amounts. For example, in some embodiments, the ketamine is present in the amount of about 2% to about 30% by weight of the gel formulation. In some embodiments, the ketamine is present in the amount of about 2%, about 2.5%, about 5%, about 10%, about 15%, about 18%, about 20%, about 25%, about 30%, or any ranges in between the recited values, by weight of the gel formulation. In some preferred embodiments, the ketamine is present in the amount of about 2% to about 5%, about 5% to about 10%, about 5% to about 15%, or about 10% to about 20%, by weight of the gel formulation. In some specific embodiments, the ketamine is present in the amount of about 2%, about 2.5%, about 5%, about 10%, about 15%, about 18% by weight of the gel formulation. The ketamine in the gel formulation can be in a racemic form, a substantially pure S-enantiomer (e.g., with less than 10% R-isomer, less than 5% R-isomer, less than 1% R-isomer, or less than 0.1% R-isomer), a substantially pure R-enantiomer (e.g., with less than 10% S-isomer, less than 5% S-isomer, less than 1% S-isomer, or less than 0.1% S-isomer), or a mixture of S- and R-isomers in any ratio.

Various solvents are suitable for use in the ketamine gel formulation. Non-limiting useful solvents include ethanol, water, propylene glycol, acetone, isopropyl alcohol, butylene glycol, dimethyl sulfoxide (DMSO), dimethyl acetamide (DMA), and combinations thereof. The amount of solvent can be about 10% to about 90%, typically about 40% to about 75% (e.g., about 40%, about 50%, about 60%, about 70%, about 75%, or any ranges between the specified values) by weight of the gel formulation. In some embodiments, the amount of solvent can be greater than 75% by weight of the gel formulation, for example, greater than 80%, greater than 85%, etc. When two or more solvents are included, the amount of solvent refers to the total amount of solvents unless otherwise obvious from context. In some embodiments, the solvent comprises ethanol. In some embodiments, the solvent comprises ethanol and DMSO. In some embodiments, the solvent comprises ethanol and propylene glycol. In some embodiments, the solvent comprises ethanol, DMSO, and propylene glycol. In some embodiments, the solvent comprises ethanol in the amount of about 40% to about 75% (e.g., about 40%, about 50%, about 60%, about 70%, about 75%, or any ranges between the specified values) by weight of the gel formulation. In some embodiments, the solvent is free or substantially free of DMSO. In some embodiments, the solvent is free or substantially free of propylene glycol.

Various skin permeation enhancers can be used in the ketamine gel formulation to enhance the skin permeability of ketamine through the skin. Non-limiting useful skin permeation enhancers include, for example, sulfoxides (e.g., dimethylsulfoxide, DMSO), Azones (e.g., laurocapram), pyrrolidones (e.g., 2-pyrrolidone, 2P), alcohols and alkanols (e.g., ethanol or decanol), glycols (e.g., propylene glycol (PG)), surfactants (e.g., Tween 80), terpenes, and combinations thereof. See, e.g., Williams et al., Adv Drug Deliv Rev. 27; 56(5):603-18 (2004). The skin permeation enhancer is typically included in the amount of about 1% to about 25% by weight of the gel formulation, for example, about 2%, about 5%, about 10%, about 15%, about 20%, about 25%, or any ranges between the specified values, by weight of the gel formulation.

The ketamine gel formulation can include one or more skin permeation enhancers. For example, in some embodiments, the permeation enhancer comprises one or more compounds chosen from sulfoxides, alcohols, alkanols, glycols, and surfactants. In some embodiments, the permeation enhancer comprises one or more compounds chosen from dimethyl sulfoxide (DMSO), oleic alcohol, oleic acid, levulinic acid, propylene glycol, dipropylene glycol, ethanol, and surfactants such as Tween 80. In some preferred embodiments, the permeation enhancer comprises one or more (e.g., 1, 2, 3, 4, or all), preferably one to three, more preferably, two or three, of levulinic acid, oleic acid, oleic alcohol, DMSO, and dipropylene glycol. In some preferred embodiments, the permeation enhancer comprises one or more (e.g., 1, 2, 3, 4, or all), preferably one to three, more preferably, two or three, of the following: (a) levulinic acid in the amount of about 0.1% to about 15% (preferably, about 1% to about 15%, such as about 1%, about 2%, about 5%, about 8%, about 10%, about 15%, or any ranges between the specified values) by weight of the gel formulation; (b) oleic acid in the amount of about 0.1% to about 15% (preferably, about 1% to about 10%, such as about 1%, about 2%, about 5%, about 8%, about 10%, or any ranges between the specified values) by weight of the gel formulation; (c) oleic alcohol in the amount of about 0.1% to about 15% (preferably, about 1% to about 10%, such as about 1%, about 2%, about 5%, about 8%, about 10%, or any ranges between the specified values) by weight of the gel formulation; (d) DMSO in the amount of about 0.1% to about 15% (preferably, about 1% to about 15%, such as about 1%, about 2%, about 5%, about 8%, about 10%, about 15%, or any ranges between the specified values) by weight of the gel formulation; and (e) dipropylene glycol in the amount of about 0.1% to about 15% (preferably, about 1% to about 15%, such as about 1%, about 2%, about 5%, about 8%, about 10%, about 15%, or any ranges between the specified values) by weight of the gel formulation.

In some preferred embodiments, the permeation enhancer comprises levulinic acid in the amount of about 5% to about 15% by weight of the gel formulation and oleic alcohol in the amount of about 1% to about 8% by weight of the gel formulation. In some embodiments, the permeation enhancer further comprises DMSO in the amount of about 5% to about 15% by weight of the gel formulation.

In some preferred embodiments, the permeation enhancer comprises levulinic acid in the amount of about 5% to about 15% by weight of the gel formulation and oleic acid in the amount of about 1% to about 8% by weight of the gel formulation.

In some preferred embodiments, the permeation enhancer comprises levulinic acid in the amount of about 1% to about 10% by weight of the gel formulation and dipropylene glycol in the amount of about 5% to about 15% by weight of the gel formulation.

While the ketamine gel formulation can include one or more of DMSO, oleic alcohol, oleic acid, levulinic acid, dipropylene glycol, and propylene glycol, in some embodiments, the ketamine gel formulation can also be free or substantially free of one or more of these compounds. For example, in some embodiments, the ketamine gel formulation can be free or substantially free of DMSO. In some embodiments, the ketamine gel formulation can be free or substantially free of oleic alcohol. In some embodiments, the ketamine gel formulation can be free or substantially free of oleic acid. In some embodiments, the ketamine gel formulation can be free or substantially free of levulinic acid. In some embodiments, the ketamine gel formulation can be free or substantially free of dipropylene glycol. In some embodiments, the ketamine gel formulation can be free or substantially free of propylene glycol. In some embodiments, the ketamine gel formulation can be free or substantially free of two or more, preferably, three or more such as 3, 4, or 5, compounds chosen from DMSO, oleic alcohol, oleic acid, levulinic acid, dipropylene glycol, and propylene glycol. In some embodiments, the ketamine gel formulation can be free or substantially free of all of DMSO, oleic alcohol, oleic acid, levulinic acid, dipropylene glycol, and propylene glycol.

Many agents can have dual or multiple functions. For example, some of the solvents described herein can also act as skin permeation enhancers, for example, ethanol, DMSO, propylene glycol, etc. For clarity of calculation of weight percentages, as used herein, when a dual (or multiple) functional agent is included in a ketamine formulation or transdermal delivery device, the amount of that agent is proper as long as it can fit into one of the specified weight limits according to one of its functions, unless otherwise obvious from context. For example, in an embodiment specifying a solvent in an amount of about 40% to about 75% by weight and a skin permeation enhancer in an amount of about 5% to about 25% by weight, a ketamine formulation including ethanol (55% by weight), DMSO (10% c by weight), and one or two permeation enhancers in a combined amount of 20% by weight can still be within that embodiment; even though counting either one of DMSO and ethanol as a permeation enhancer would violate the upper limit specified for the permeation enhancer, both DMSO and ethanol can act as a solvent and the total amount of ethanol and DMSO are within the limits specified for the solvent. It should be clear that the total amount of ingredients should not exceed 100% and the same agent is calculated only once towards the total amount. In some embodiments, the gel formulation comprises a non-solvent skin permeation enhancer in the amount of about 1% to about 25% by weight of the gel formulation, for example, about 2%, about 5%, about 10%, about 15%, about 20%, about 25%, or any ranges between the specified values, by weight of the gel formulation.

Various gel forming agents are suitable for the ketamine gel formulation herein. Non-limiting useful gel forming agents include, for example, a hydroxypropylcellulose (such as Klucel HF Pharm), hydroxypropyl methyl cellulose (HPMC), polyvinyl pyrolidone (PVP such as BASF's Kollidon), polyacrylic acid (such as Carbopol), sodium CMC (carboxyl methyl cellulose), and combinations thereof. In some specific embodiments, the gel forming agent is a hydroxypropylcellulose, such as commercially available ones including Klucel's hydroxypropylcellulose with a grade of HF Pharm. The amount of gel forming agent can vary so long as it is present in an amount sufficient to cause gel formation. Typically, the gel forming agent is present in an amount of about 0.1% to about 20% (e.g., about 1%, about 2%, about 3%, about 4%, about 5%, about 10%, about 15%, about 20%, or any ranges between the specified values) by weight of the gel formulation. In some embodiments, the gel-forming agent is included such that the ketamine gel formulation has a viscosity (Brookfield viscosity) of about 15,000 cP to about 45,000 cP.

In some embodiments, the gel forming agent includes polyvinyl alcohol with a molecular weight ranging from about 20,000-200,000, specific gravity ranging from about 1.19-1.31, and viscosity ranging from about 4-65 cps. The polyvinyl alcohol used in the formulation is preferably a water-soluble synthetic polymer represented by —(—C₂H₄O—)ₙ—, where n can range from about 500-5,000. Examples of suitable, commercially available polyvinyl alcohol polymers include PVA, USP, available from Spectrum Chemical Manufacturing Corporation, New Brunswick, N.J. 08901.

In some embodiments, the gel forming agent includes hydroxypropyl methyl cellulose (Hypromellose) with a molecular weight ranging from about 10,000-1,500,000, typically from about 5000-10,000 (i.e., low molecular). The specific gravity ranges from about 1.19-1.31, with an average specific gravity of about 1.26. Viscosity is about 3600-5600 cPs. The hydroxypropyl methylcellulose used in the formulation can be a water-soluble synthetic polymer. Examples of suitable, commercially available hydroxypropyl methylcellulose polymers include Methocel K100 LV and Methocel K4M, available from Dow chemicals.

In some embodiments, the gel forming agent includes carbomers, having a molecular weight ranging from 700,000-4,000,000 and viscosity ranging from about 4000-39,400 cPs. Examples of suitable, commercially available carbomers include carbopol 934P NF, carbopol 974P NF, and carbopol 971P NF, available from Lubrizol.

Other suitable excipients useful in the preparation of transdermal delivery devices such as humectants, plasticizers, antioxidants, anti-irritants, etc. can also be included in the ketamine gel formulation herein. These excipients are within the knowledge of those skilled in the art, and can be found, for example, in the Handbook of Pharmaceutical Excipients, (7^{th} ed. 2012), the entire content of which is hereby incorporated by reference. For example, in some embodiments, the ketamine gel formulation can optionally include an anti-irritant such as aloe, arnica, chamomile, cucumber, menthol, mugwort, oat, zinc oxide, a humectant, a plasticizer, an antioxidant, and/or a drug release modifier such as chitosan, cellulose-based polymers, silicon dioxides, and polymethacrylates.

For example, in any of the embodiments described herein, the ketamine gel formulation can include an antioxidant agent. In some embodiments, the antioxidant agent is present in the amount of about 0.001% to about 2% (e.g., about 0.001%, about 0.005%, about 0.01%, about 0.05%, about 0.1%, about 1%, about 1.5%, about 2%, or any ranges between the specified values) by weight of the gel formulation. Non-limiting useful antioxidants include, but are not limited to, butylated hydroxyanisole (BHA), butylhydroxy toluene (BHT), tert-Butylhydroquinone, ascorbic acid, and tocopherols. In some embodiments, inclusion of one or more antioxidant agents can help stabilizing the gel formulation from oxidative degradation of ketamine.

Specific Ketamine Gel Formulations

Certain embodiments of the present disclosure are directed to some specific ketamine gel formulations. Table 1 below shows some of the specific ketamine gel formulations according to embodiments of the present disclosure. All weight percentages in Table 1 are based on total weight of the ketamine gel formulation, and should be understood as preceded by the word “about”. Further, it should be understood that in preferred embodiments, the ketamine gel formulation of Table 1 should include at least two or more (e.g., 2, 3, or 4) of DMSO, oleic alcohol, oleic acid, levulinic acid, dipropylene glycol, and propylene glycol, i.e., two of more of these ingredients are not 0%. More preferably, the total amount of the at least two or more (e.g., 2, 3, or 4) of DMSO, oleic alcohol, oleic acid, levulinic acid, dipropylene glycol, and propylene glycol is about 15% to about 45% (preferably about 20% to about 40%, such as about 25% to about 35%) by weight of the ketamine gel formulation. In some embodiments, the ketamine gel formulation described herein is free or substantially free of one or more of DMSO, oleic alcohol, oleic acid, levulinic acid, dipropylene glycol, and propylene glycol.

TABLE 1
Exemplary Ketamine Gel Formulation

| | | | More | |
|---|---|---|---|---|
| | Typical | Preferred | Preferred | Exemplary |
| Ingredient | wt % | wt % | wt % | wt % |
| Ketamine base | 2-30% | 2-20% | 5-15%, | 2.5%, 5%, 10%, |
| | | | 5-10% | 15%, 18% |
| Alcohol USP | 40-75% | 45-70% | 50-65% | 47.8%, 57.8% |
| (ethanol) | | | | |
| DMSO | 0.1-15% or | 1-15% or | 5-15% or | 10%, 0% |
| | 0% | 0% | 0% | |
| Oleic alcohol | 0.1-15% or | 1-10% or | 1-8% or | 5%, 0% |
| | 0% | 0% | 0% | |
| Oleic acid | 0.1-15% or | 1-10% or | 1-8% or | 5%, 0% |
| | 0% | 0% | 0% | |
| Levulinic acid | 0.1-15% or | 1-15% or | 5-15% or | 0%, 5%, 10% |
| | 0% | 0% | 0% | |
| Dipropylene | 0.1-15% or | 1-15% or | 5-15% or | 10%, 0% |
| glycol | 0% | 0% | 0% | |
| Propylene | 0.1-15% or | 1-15% or | 5-15% or | 10%, 0% |
| glycol | 0% | 0% | 0% | |
| Gel forming | 0.1-5% | 0.5-5% | 1-5% | 2% |
| agent (e.g., | | | | |
| Klucel HF | | | | |
| Pharm) | | | | |
| TOTAL | 100% | 100% | 100% | 100% |

In some embodiments, the ketamine gel formulation includes about 15% ketamine by weight. Table 2 below shows some specific ketamine gel formulations with a 15% ketamine concentration according to embodiments of the present disclosure. All weight percentages in Table 2 are based on total weight of the ketamine gel formulation, and should be understood as preceded by the word “about”.

TABLE 2
Exemplary 15% Ketamine Gel Formulation

| | 1 | 2 | 3 | 4 | 5 |
|---|---|---|---|---|---|
| Ingredient | wt % | wt % | wt % | wt % | wt % |
| Ketamine base | 15.0% | 15.0% | 15.0% | 15.0% | 15.0% |
| Alcohol USP | 57.8% | 60.0% | 57.8% | 57.8% | 47.8% |
| DMSO | | | | | 10.0% |
| Oleic alcohol | 5.0% | 5.0% | | | 5.0% |
| Oleic acid | | | 5.0% | | |
| Levulinic acid | 10.0% | 9.0% | 10.0% | 5.0% | 10.0% |
| Dipropylene | | | | 10.0% | |
| glycol | | | | | |
| Propylene glycol | 10.0% | 9.0% | 10.0% | 10.0% | 10.0% |
| Klucel HF Pharm | 2.0% | 2.0% | 2.0% | 2.0% | 2.0% |
| TOTAL | 100% | | 100% | 100% | 100% |

In some embodiments, the ketamine gel formulation includes about 10% ketamine by weight. Table 3 below further shows some specific ketamine gel formulations with a 10% ketamine concentration according to embodiments of the present disclosure. All weight percentages in Table 3 are based on total weight of the ketamine gel formulation, and should be understood as preceded by the word “about”.

TABLE 3
Exemplary 10% Ketamine Gel Formulation

| | 1 | 2 | 3 | 4 | 5 |
|---|---|---|---|---|---|
| Ingredient | wt % | wt % | wt % | wt % | wt % |
| Ketamine base | 10.0% | 10.0% | 10.0% | 10.0% | 10.0% |
| Alcohol USP | 64.8% | 67.0% | 64.8% | 64.8% | 54.8% |
| DMSO | | | | | 10.0% |
| Oleic alcohol | 5.0% | 5.0% | | | 5.0% |
| Oleic acid | | | 5.0% | | |
| Levulinic acid | 8.0% | 7.0% | 8.0% | 5.0% | 8.0% |
| Dipropylene | | | | 8.0% | |
| glycol | | | | | |
| Propylene glycol | 10.0% | 9.0% | 10.0% | 10.0% | 10.0% |
| Klucel HF Pharm | 2.0% | 2.0% | 2.0% | 2.0% | 2.0% |
| TOTAL | 100% | | 100% | 100% | 100% |

Ketamine Patch Comprising Ketamine Gel Formulation

The ketamine gel formulation described herein can be included in a transdermal delivery device, preferably a gel reservoir-type DIR transdermal patch. The structure and packaging of the transdermal delivery device comprising the ketamine gel formulation herein can be prepared in accordance with methods described herein and techniques known to persons skilled in the art. In preferred embodiments, the transdermal delivery device (e.g., DIR patches described herein) is configured to achieve the skin flux characteristics described hereinabove. However, it should be understood that in some embodiments, the transdermal delivery device (e.g., a DIR patch) comprising any of the ketamine gel formulation described herein is also a novel aspect of the present disclosure and can be independent of the above skin flux characteristics. Thus, some embodiments of the present disclosure are also directed to the transdermal delivery device.

Typically, the transdermal delivery device includes a backing layer, a reservoir layer comprising any of the ketamine gel formulation described herein, an adhesive layer, and a release liner. The reservoir layer and the adhesive layer are typically two separate layers that are laminated to each other or separated, for example, by a rate-controlling membrane. In some embodiments, the adhesive layer is a separate layer configured to contact the skin of a human subject and the reservoir layer is in between the adhesive layer and the backing layer.

In some embodiments, the transdermal delivery device is a DIR patch comprising a ketamine gel formulation. Typically, the DIR patch includes a backing layer, a reservoir layer comprising any of the ketamine gel formulation described herein, a rate-controlling membrane, an adhesive layer, and a release liner. In preferred embodiments, the DIR patches described herein are configured to achieve the skin flux characteristics described hereinabove for the transdermal delivery device.

The reservoir layer of the transdermal delivery device (e.g., DIR patch) can comprise, consist essentially of, or consist of any of the ketamine gel formulation described herein. Preferably, the ketamine gel formulation is used such that the transdermal delivery device (e.g., DIR patch), when applied to the skin of a human subject, causes minimal or no skin irritation. The reservoir layer in a DIR patch is generally sandwiched between a backing film and a rate-controlling membrane such as a microporous membrane (see, e.g., FIG. 1).

The weight and thickness of the reservoir layer can vary depending on different factors such as drug concentration and desired duration of administration, etc. In some embodiments, the reservoir layer can have a gel weight of about 0.15 g/cm² to about 0.90 g/cm² (e.g., about 0.15 g/cm² to about 0.24 g/cm²) active surface area. In some embodiments, the reservoir layer can have a thickness of about 1.5 mm to about 3.5 mm (e.g., about 2 mm to about 3.5 mm). In some embodiments, the reservoir layer can comprise an amount of ketamine sufficient to provide about 0.1 mg/day/cm² to about 30 mg/day/cm² (e.g., about 1 mg/day/cm², about 2 mg/day/cm², about 5 mg/day/cm², about 10 mg/day/cm², about 15 mg/day/cm², about 20 mg/day/cm², or any ranges between the specified values) of ketamine over a period of time selected from about 8 hours, about 12 hours, about 18 hours, about 24 hours, about 2 days, about 3 days, about 4 days, about 5 days, about 6 days, and about 7 days. For example, in some embodiments, the reservoir layer can comprise an amount of ketamine sufficient to provide about 1 mg/day/cm² to about 10 mg/day/cm² (preferably, about 1 mg/day/cm² to about 5 mg/day/cm² or about 2 mg/day/cm² to about 10 mg/day/cm²) of ketamine over a period of time selected from about 24 hours, about 2 days, about 3 days, about 4 days, about 5 days, about 6 days, and about 7 days. In some embodiments, the reservoir layer can comprise an amount of ketamine sufficient to provide about 0.1 mg/day/cm² to about 30 mg/day/cm², preferably, about 1 mg/day/cm² to about 10 mg/day/cm² (preferably, about 1 mg/day/cm² to about 5 mg/day/cm² or about 2 mg/day/cm² to about 10 mg/day/cm²) of ketamine over a period of more than 7 days. In some embodiments, the reservoir layer can comprise an amount of ketamine sufficient to provide about 0.1 mg/day/cm² to about 30 mg/day/cm², preferably, about 1 mg/day/cm² to about 10 mg/day/cm² (preferably, about 1 mg/day/cm² to about 5 mg/day/cm² or about 2 mg/day/cm² to about 10 mg/day/cm²) of ketamine over a period of less than 24 hours such as less than 18 hours, less than 12 hours, less than 8 hours, or less than 4 hours.

The rate-controlling membrane, if present, is typically a microporous membrane. Non-limiting useful microporous membrane include polypropylene films (e.g., Celgard 2400, Celgard 2500), poly ethylene vinyl acetate (EVA) films (e.g., CoTran 9702, CoTran 9715), and combinations thereof. A skilled person could select proper microporous membrane to adjust ketamine flux of the transdermal delivery device in view of the present disclosure and background techniques.

The adhesive layer typically includes a pressure sensitive adhesive (PSA). PSAs are generally known in the art. See, e.g., Tan et al., Pharm Sci & Tech Today, 2:60-69 (1999). Non-limiting useful PSAs include polyisobutylenes (PIB), silicone polymers, acrylate copolymers, and combinations thereof. In some embodiments, the pressure sensitive adhesive comprises a polyisobutylene adhesive, a silicone polymer adhesive, an acrylate copolymer adhesive, or a combination thereof. In some embodiments, the pressure sensitive adhesive comprises an acrylate copolymer adhesive. Non-limiting useful acrylate copolymers include, for example, acrylic pressure sensitive adhesives such as a poly acrylate vinyl acetate copolymer, e.g., Duro-Tak 87-2287, Duro-Tak 87-4098, Duro-Tak 87-4287, or Duro-Tak 87-2516, Duro-Tak 87-2852 or Duro-Tak 87-2194,), which are manufactured by Henkel Adhesives. PIBs are elastomeric polymers that are commonly used in PSAs, both as primary-base polymers and as tackifiers. PIBs are homopolymers of isobutylene and feature a regular structure of a carbon-hydrogen backbone with only terminal unsaturation. Non-limiting useful PIBs include those marketed under the trade name Oppanol by BASF. The silicone polymers are a high molecular weight polydimethylsiloxane that contains residual silanol functionality (SiOH) on the ends of the polymer chains. Non-limiting useful silicone PSAs for use in pharmaceutical applications include those available from Dow Corning Corporation, for example under the trade name of BIO-PSA. In some embodiments, the adhesive layer is about 1.5 mils to about 10 mils (e.g., about 1.5 mils to about 2 mils) thick.

The transdermal delivery devices (e.g., DIR patches) herein can have different sizes (patch sizes) depending on its application. Typically, the patch sizes can be about 5 cm² to about 300 cm² (e.g., about 5 cm², about 10 cm², about 20 cm², about 30 cm², about 40 cm², about 50 cm², about 60 cm², about 80 cm², about 100 cm², about 120 cm², about 150 cm², about 200 cm² or any ranges between the specified values), for example, about 10 cm² to about 100 cm². For a typical DIR patch, the patch size refers to the area of the reservoir portion of adhesive surface (normally in the central portion of the patch).

When applying the transdermal delivery devices (e.g., DIR patches) herein to a skin of a subject, all of the adhesive surface can become in contact with the skin in theory. Thus, the area of the adhesive surface defines a contact area, i.e., a skin contact area, which is also referred herein to as an active surface area. The area of the adhesive surface can determine the doses of ketamine to be delivered. Typically, the area of the adhesive surface can be about 5 cm² to about 300 cm² (e.g., about 5 cm², about 10 cm², about 20 cm², about 30 cm², about 40 cm², about 50 cm², about 60 cm², about 80 cm², about 100 cm², about 120 cm², about 150 cm², about 200 cm² or any ranges between the specified values), for example, about 10 cm² to about 100 cm². In some embodiments, the adhesive surface is the only surface of the transdermal delivery device that is in contact with the skin upon application, and the active surface area is the same as the area of the adhesive surface.

In some embodiments, the adhesive surface and one or more other surfaces of the transdermal delivery device can be in contact with the skin upon application, and the entire skin contact area is the active surface area. In any of the embodiments described herein, the active surface area can be about 5 cm² to about 300 cm² (e.g., about 5 cm², about 10 cm², about 20 cm², about 30 cm², about 40 cm², about 50 cm², about 60 cm², about 80 cm², about 100 cm², about 120 cm², about 150 cm², about 200 cm² or any ranges between the specified values), for example, about 10 cm² to about 100 cm².

Typically, the transdermal delivery device (e.g., a DIR patch) is supported by an impermeable backing film, and the adhesive surface is protected by a release liner. Various materials can be used as a backing layer for the transdermal delivery device herein. Typically, the backing layer is impermeable. For example, the backing layer can be comprised of impermeable polymeric films such as polyester (PET) or polyethylene (PE) films. In some embodiments, the backing layer can comprise a polyester, such as Scotchpak 9736 or Scotchpak 1012, a polyurethane film, such as Scotchpak 9701, or a polyethylene film, such as CoTran 9720.

The release liner can be manufactured in the desired size for the present invention. The release liner may be comprised of silicone or fluoro-polymer coated polyester film. The release liner protects the transdermal delivery device during storage and is removed before its use. Silicone-coated release liners are manufactured by Mylan Corporation, Loparex Corporation, and 3M's Drug Delivery Systems. The fluoro-polymer coated release liners are manufactured and supplied by 3M's Drug Delivery Systems and Loparex. In some embodiments, the release liner comprises 3M's ScotchPak 9744 or Scotchpak 1022.

The transdermal delivery device described herein can optionally include additional active agent(s) other than ketamine. Such additional drug(s) can be incorporated, for example, to counteract adverse effects, and/or to enhance the antidepressant and/or pain management effect of ketamine. Exemplary drugs for enhancing antidepressant effect include, but are not limited to, antagonists of group II metabotropic glutamate receptors, such as LY341495, Podkowa et al., Psychopharmacology (Berl) (Jun. 11, 2016). Exemplary drugs for reducing side effects with ketamine, especially psychotomimetic and sympathomimetic, include, but not limited to, alpha-2 agonists such as clonidine, Lenze, World J Biol Psychiatry, 17(3):230-8 (2016). However, in any one of the embodiments described herein, ketamine can also be the only active agent included in the ketamine gel formulation or the transdermal delivery device described herein.

Abuse Deterrent

Ketamine is also a known dissociative anesthetic that has gained popularity as a drug of abuse, and may be referred to illicitly as “K” or “Special K”. Ketamine is reported to distort perceptions of sight and sound, and make the user feel disconnected. The 2011 “Monitoring the Future” (MTF) study reports the annual use of ketamine among 8^{th}, 10^{th}, and 12^{th} graders as being 0.8%, 1.2%, and 1.7%, respectively. Johnston, et al., 2012. Monitoring the future national results on adolescent drug use: Overview of key findings, 2011, Ann Arbor: Institute for Social Research. The University of Michigan. Illicit ketamine can be distributed as a dried powder or as a liquid, mixed with beverages, and/or added to smokable materials (such as marijuana or tobacco). As a powder, ketamine can be snorted or pressed into tablets, sometimes in combination with other drugs, including 3,4-methylenedioxymethamphetamine (MDMA, referred to illicitly as “ecstasy”), amphetamine, methamphetamine, cocaine, and/or carisoprodol. On Aug. 12, 1999 ketamine became a Schedule 111 non-narcotic substance under the Controlled Substances Act. Consequently, there is a need to develop abuse deterrent mechanisms to reduce the risk of ketamine abuse.

The ketamine gel formulation herein or transdermal delivery device comprising the ketamine gel formulation can be potentially abused. Thus, in some embodiments, the ketamine gel formulation herein or transdermal delivery device further includes one or more abuse deterrent agents. Typically, the one or more abuse deterrent agents are included in the reservoir layer and/or adhesive layer of the transdermal delivery device described herein. However, in some embodiments, the one or more abuse deterrent agents can also be included in a separate layer. In some embodiments, the one or more abuse deterrent agents are selected from aversive agents, such as capsaicin, apomorphine, denatonium, sodium laurel sulfate, niacin and combinations thereof.

Generally, abuse deterrent agents are employed because they have one or more of the following properties: (1) unpalatable bitterness or other repulsive tastes in the mouth (i.e., bittering agents); (2) formation of gel upon mixing with the extraction solvents (i.e., gel forming agents); (3) severe irritation when injected (i.e., irritants); (4) mood depression (e.g., droperidol) or other pronounced central nervous system (CNS) effects; (5) acute gastrointestinal, cardiac or respiratory effects; (6) violent nausea or vomiting; (7) repugnant smells if not used as instructed; (8) sleep inducing, thereby causing the abuser to miss or be made unaware of the euphoria; (9) deactivate or degrade the active ingredient (i.e., strong oxidation agents (such as hydrogen peroxide), strong acid, or strong base, and/or antagonists) upon attempted extraction.

Bittering agents are pharmaceutically acceptable bitter substances that create a bitter taste or effect when administered nasally (snorted), orally, buccally or sublingually, making consumption difficult. The bittering agents that may be employed in the present invention include, but are not limited to, sucrose octaacetate (used as a denaturant for alcohol) (e.g., SD-40), denatonium saccharide, denatonium benzoate, caffeine, quinine (or a quinine salt such as quinine sulfate), bitter orange peel oil, and other botanical extract ingredients, such as pepper extract (cubeb), capsicum, and the like. Preferred bittering agents are sucrose octaacetate, denatonium benzoate (Bitrex), and denatonium saccharide (four times more bitter than denatonium benzoate) because they are extremely bitter even at low concentrations and essentially non-toxic.

In some embodiments, the one or more abuse deterrent drugs can include one or more irritants. Irritants are pharmaceutically inert compounds that induce irritation to the mucous membranes of the body (i.e., nasal, mouth, eye, intestine, urinary tract). The irritants that may be employed in the present invention include, but are not limited to surfactants, such as sodium lauryl sulfate (SLS), poloxamer, sorbitan monoesters and glyceryl monooleates, as well as spicy ingredients, and others.

In embodiments of the present invention, the irritant can deter abuse when the transdermal delivery device is tampered with. For example, if an abuser extracts and dries the ketamine, then the irritant is exposed, discouraging inhalation of the ketamine mixed with the irritant, as inhalation (e.g., via snorting through the nose) will induce pain and/or irritation of the abuser's mucous membrane and/or nasal passageway tissue.

The nature of the ketamine gel formulation herein or the transdermal delivery device comprising the ketamine gel formulation can provide some abuse deterrent properties. Without wishing to be bound by theories, because the ketamine gel formulation includes one or more gel forming agents, which upon contact with an extracting solvent (e.g., water or alcohol), the one or more gel forming agents can absorb the extracting solvent and swell, thereby significantly reduces and/or minimizes the amount of free solvent which can contain an amount of solubilized ketamine, and which minimizes what can be drawn into a syringe for injection (i.e., IV or intramuscular).

In some embodiments, in addition to the gel forming agent(s) in the ketamine gel formulation, additional gel forming agent(s) can be included, for example, in a segregated layer, e.g., laminated to the reservoir layer, to further deter abuse potentials. Suitable gel forming agents are described herein. In some embodiments, the ketamine gel formulation or the transdermal delivery device can also be free or substantially free of an abuse deterrent drug that is not a gel forming agent. In some embodiments, the ketamine gel formulation or the transdermal delivery device can also be free or substantially free of the one or more abuse deterrent drugs described herein, which are not gel forming agents.

Stability

The transdermal delivery device described herein is preferably storage stable. As used herein, a “storage stable” transdermal delivery device can be (1) a device that is free or substantially free of crystals (e.g., drug related crystals) and/or (2) a device that maintains or substantially maintains the skin flux characteristics when tested in vitro using human cadaver skin, after being stored at 25° C., 60% relative humidity, for a given period of time, for example, for at least 4 weeks (e.g., at least 1 month, at least 2 months, at least 3 months, at least 4 months, at least 5 months, at least 6 months, etc.). Preferably, the transdermal delivery device described herein satisfies both (1) and (2).

Drug crystallization will retard drug release and skin permeability, reducing the efficacy of the transdermal delivery device. Drug crystals should preferably not be formed in the transdermal delivery device over a period approximating the shelf life, e.g., for about 6 months or greater. Preferably, the transdermal delivery device is free or substantially free of crystals (e.g., drug related crystals) after being stored at 25° C., 60% relative humidity, for about 1 month, about 2 months, about 3 months, about 4 months, about 5 months, about 6 months, or longer.

Typically, a crystallization inhibitor such as polyvinyl pyrrolidone-co-vinyl acetate and/or polymethacrylate is added to a formulation to prevent crystallization. However, the inventors have found that the transdermal delivery device comprising the ketamine gel formulation described herein, even without a crystallization inhibitor, can be free or substantially free of crystals (e.g., drug related crystals) after being stored at 25° C., 60% relative humidity, for 5 months or longer. Thus, in some embodiments, the present disclosure also provides a transdermal delivery device comprising the ketamine gel formulation described herein, which is free or substantially free of a crystallization inhibitor. In some embodiments, the present disclosure provides a transdermal delivery device comprising the ketamine gel formulation described herein, which is free or substantially free of a crystallization inhibitor selected from polyvinyl pyrrolidone-co-vinyl acetate, polymethacrylate, and combinations thereof.

In some embodiments, the transdermal delivery device described herein maintains or substantially maintains its flux characteristics when tested in vitro using human cadaver skin, after being stored at 25° C., 60% relative humidity, for about 1 month, about 2 months, about 3 months, about 4 months, about 5 months, about 6 months, or longer. For example, in some embodiments, upon storage at 25° C., 60% relative humidity, for 5 months after preparation, the transdermal delivery device provides one or more of the following skin flux characteristics when tested in vitro using human cadaver skin: (a) a cumulative ketamine permeated at 24 hours post administration that is within the range of about 50% to about 250% of that observed for the same transdermal delivery device stored at 25° C. for 1 week after preparation; (b) a cumulative ketamine permeated at 48 hours post administration that is within the range of about 60% to about 160% of that observed for the same transdermal delivery device stored at 25° C. for 1 week after preparation; and (c) a cumulative ketamine permeated at 72 hours post administration that is within the range of about 75% to about 160% of that observed for the same transdermal delivery device stored at 25° C. for 1 week after preparation. For example, in some embodiments, upon storage at 25° C., 60% relative humidity, for 5 months after preparation, the transdermal delivery device provides one or more of the following skin flux characteristics when tested in vitro using human cadaver skin: (a) a cumulative ketamine permeated at 24 hours post administration that is the same or substantially the same (e.g., within the range of about 80% to about 125%) as that observed for the same transdermal delivery device stored at 25° C. for 1 week after preparation; (b) a cumulative ketamine permeated at 48 hours post administration that is the same or substantially the same (e.g., within the range of about 80% to about 125%) as that observed for the same transdermal delivery device stored at 25° C. for 1 week after preparation; and (c) a cumulative ketamine permeated at 72 hours post administration that is the same or substantially the same (e.g., within the range of about 80% to about 125%) as that observed for the same transdermal delivery device stored at 25° C. for 1 week after preparation.

Methods of Administering Ketamine

Certain embodiments of the present disclosure are directed to methods of administering ketamine to a subject (e.g., a human subject) in need thereof. In some embodiments, the method is for antagonizing NMDA receptors in the subject. In some embodiments, the method is for treating a disease or disorder in the subject, where antagonizing NMDA receptor is beneficial. In some embodiments, the method is for treating depression, anxiety, and/or pain in the subject. In some embodiments, the subject is characterized as having depression (e.g., a major depressive disorder). In some embodiments, the subject is characterized as having pain (e.g., neuropathic pain). In some embodiments, the disease or disorder is one or more chosen from pain (e.g., neuropathic pain, complex regional pain syndrome (CRPS), chronic pain), depression (major depressive disorder, treatment-resistant depression, bipolar depression), restless legs syndrome, a condition associated with spinal cord injury (e.g., autonomic dysreflexia, immune suppression, chronic central neuropathic pain suffering from spinal cord injury, leukocyte apoptosis, splenic atrophy, leucopenia, or combinations thereof), anxiety, bipolar disorder (e.g., childhood-onset bipolar disorder, bipolar depression), stress-induced disorder (e.g., stress-induced affective disorder, stress-induced psychopathology), post-traumatic stress disorder, Alzheimer's dementia, amyotrophic lateral sclerosis, and suicidality.

In some embodiments, the method comprises applying a transdermal ketamine formulation, such as in a transdermal delivery device described herein, to the skin of the subject. In any of the embodiments described herein, the method can deliver to the subject about 0.1 mg/day/cm² to about 30 mg/day/cm², preferably, about 1 mg/day/cm² to about 10 mg/day/cm² (preferably, about 1 mg/day/cm² to about 5 mg/day/cm² or about 2 mg/day/cm² to about 10 mg/day/cm²) of ketamine, for example, for a period of time selected from about 8 hours, about 12 hours, about 18 hours, about 24 hours, about 2 days, about 3 days, about 4 days, about 5 days, about 6 days, and about 7 days. In some embodiments, applying the transdermal ketamine formulation provides a therapeutically effective concentration of ketamine for an extended period. In some embodiments, applying the transdermal ketamine formulation provides a lower Cₘₐₓ compared to that of a dose-equivalent intravenous or intranasal formulation. In some embodiments, the transdermal ketamine formulation can be any of the ketamine gel formulation or the transdermal delivery device described herein. In some embodiments, the transdermal ketamine formulation can also be, for example, any of the transdermal delivery devices described in International Application No. PCT/US2016/039601, published as WO2017/003935, the content of which is hereby incorporated by reference in its entirety.

Typically, for any of the methods described hereinabove, the transdermal ketamine formulation is applied to provide a therapeutically effective concentration of ketamine in the plasma of the subject for a desired period of time. The therapeutically effective concentration of ketamine can vary, typically ranges from about 0.1 ng/ml to about 1500 ng/ml, preferably, about 1 ng/ml to about 1000 ng/ml. In some embodiments, the subject is characterized as having a depression (e.g., a major depressive disorder). In such embodiments, the therapeutically effective concentration of ketamine can be controlled to achieve an antidepressant effect (e.g., the treatment of MDD). For example, the plasma concentration in the subject can be controlled to range from about 10 ng/ml to about 200 ng/ml, preferably, range from about 20 ng/ml to about 100 ng/ml.

In some embodiments, the subject is characterized as having pain (e.g., a neuropathic pain). In such embodiments, the therapeutically effective concentration of ketamine can be controlled to achieve pain management. For example, the plasma concentration in the subject can be controlled to range from about 50 ng/ml to about 1000 ng/ml, and preferably from about 100 ng/ml to about 1000 ng/ml or from about 300 ng/ml to about 1000 ng/ml, such as about 500 ng/ml.

In various embodiments, the present inventors found that applying the transdermal delivery device described herein can provide an initial slow-rising pharmacokinetics and/or a sustain level of ketamine over an extended period of time, which can offer advantages, for example, over ketamine administration through conventional intravenous infusion. Accordingly, in some embodiments, the present disclosure provides a method of administering ketamine to a subject in need thereof, the method comprising applying any of the ketamine gel formulation or transdermal delivery device described herein to the skin of the subject. In some embodiments, the method is for (1) antagonizing NMDA receptors; (2) treating a disease or disorder where antagonizing NMDA receptor is beneficial; and/or (3) treating depression, anxiety, and/or pain in the subject in need thereof. In some embodiments, the method comprises applying any of the transdermal delivery devices described herein to the skin of the subject. The transdermal delivery device is typically applied according to normal practice such that all or substantially all of the active surface area of the transdermal delivery device is in contact with the skin of the subject (e.g., human subject).

In some embodiments, the transdermal delivery device is applied to provide a pharmacokinetic (“PK”) profile in the subject (e.g., human subject) characterized by (1) an initial slow-rising phase; and/or (2) a sustained release phase with ketamine concentrations being substantially constant (e.g., with fluctuation within about 0.5 to about 2 fold relative to the mean value). As used herein, a slow-rising phase refers to a first time period, which is from the application of the transdermal delivery device up to a time point when either Cₘₐₓ is reached or when the ketamine plasma concentration becomes substantially constant for a second time period after that time point. As used herein, a sustained release phase refers to the second time period during which the ketamine plasma concentration is substantially constant. The precise time point separating the slow-rising phase and the sustained release phase is not critical, and a skilled person could readily determine whether a given time is within the slow-rising phase or the sustained release phase.

In some embodiments, the initial slow-rising phase lasts until about 18 hours post application. In some embodiments, the PK profile during the initial slow-rising phase is such that the ketamine concentration in plasma does not reach peak before about 18 hours or before about 24 hours post application. In some embodiments, the PK profile during the initial slow-rising phase is such that the pharmacokinetic profile can be characterized by a ratio of ketamine concentrations in plasma at 24 hours and 12 hours post application, C_{24 h}/C_{12 h}, of about 3 to about 20; a ratio of ketamine concentrations in plasma at 18 hours and 12 hours post application, C_{18 h}/C_{12 h}, of about 3 to about 20; and/or a ratio of ketamine concentrations in plasma at 12 hours and 8 hours post application, C_{12 h}/C_{8 h}, of about 2 to about 10. In some embodiments, the maximum ketamine concentration during the initial slow-rising phase can range from about 0.1 ng/ml to about 1500 ng/ml, preferably, about 1 ng/ml to about 1000 ng/ml. In some embodiments, the subject is characterized as having a depression (e.g., a major depressive disorder), and the maximum ketamine concentration during the initial slow-rising phase can be controlled to range from about 10 ng/ml to about 200 ng/ml, preferably, range from about 20 ng/ml to about 100 ng/ml. In some embodiments, the subject is characterized as having pain (e.g., a neuropathic pain), and the maximum ketamine concentration during the initial slow-rising phase can be controlled to range from about 50 ng/ml to about 1000 ng/ml, and preferably from about 100 ng/ml to about 1000 ng/ml or from about 300 ng/ml to about 1000 ng/ml, such as about 500 ng/ml.

In some embodiments, the sustained release phase starts from about 18 hours post application. For example, the sustained release phase can include a time period from about 18 hours to about 24 hours, from 18 hours to about 48 hours, from 18 hours to about 72 hours, from 24 hours to about 48 hours, from about 24 hours to about 72 hours, or about 24 hours to about 196 hours, post application. In some embodiments, the PK profile during the sustained release phase can be characterized by a ratio of ketamine concentrations in plasma at 24 hours and 48 hours post application, C_{24 h}/C_{48 h}, of about 0.5 to about 1.5; and/or a ratio of ketamine concentrations in plasma at 24 hours and 18 hours post application, C_{24 h}/C_{18 h}, of about 0.5 to about 1.5. In some embodiments, the ketamine concentration during the sustained release phase can range from about 0.1 ng/ml to about 1500 ng/ml, preferably, about 1 ng/ml to about 1000 ng/ml. In some embodiments, the subject is characterized as having a depression (e.g., a major depressive disorder), and the ketamine concentration during the sustained release phase can be controlled to range from about 10 ng/ml to about 200 ng/ml, preferably, range from about 20 ng/ml to about 100 ng/ml. In some embodiments, the subject is characterized as having pain (e.g., a neuropathic pain), and the ketamine concentration during the sustained release phase can be controlled to range from about 50 ng/ml to about 1000 ng/ml, and preferably from about 100 ng/ml to about 1000 ng/ml or from about 300 ng/ml to about 1000 ng/ml, such as about 500 ng/ml.

While a sustained release phase is desirable in certain circumstances, some embodiments of the present disclosure are also directed to a method of administering ketamine without such sustained release phase. For example, in some embodiments, the administration of ketamine comprises applying a transdermal ketamine formulation (e.g., any of the transdermal delivery devices described herein) to the skin of a subject in need thereof for less than 18 hours, e.g., less than 12 hours, less than 8 hours, or less than 4 hours, such that a sustained release phase is not reached.

In some specific embodiments, the method described herein comprises applying a transdermal delivery device (e.g., described herein) to the skin of a human subject, wherein the transdermal delivery device includes a backing layer; a reservoir layer comprising ketamine in an amount of about 2% to about 30% by weight of the reservoir layer; and an adhesive layer defining an active surface area, wherein the active surface area is about 5 cm² to about 200 cm², and applying the transdermal delivery device to the skin provides a pharmacokinetic profile in the human subject characterized by one or more of the following: 1) ketamine concentration in plasma at 24 hours post application is about 5 ng/ml to about 1000 ng/mL; 2) ketamine concentration in plasma at 48 hours post application is about 6 ng/ml to about 1000 ng/mL; 3) ketamine concentration in plasma at 72 hours post application is about 3 ng/ml to about 1000 ng/mL; 4) a ratio of ketamine concentrations in plasma at 24 hours and 48 hours post application, C_{24 h}/C_{48 h}, is about 0.5 to about 1.5; 5) a ratio of ketamine concentrations in plasma at 24 hours and 12 hours post application, C_{24 h}/C_{12 h}, is about 3 to about 20; 6) a ratio of ketamine concentrations in plasma at 18 hours and 12 hours post application, C_{18 h}/C_{12 h}, is about 3 to about 20; 7) a ratio of ketamine concentrations in plasma at 12 hours and 8 hours post application. C_{12 h}/C_{8 h}, is about 2 to about 10; and 8) ketamine concentration in plasma does not reach peak before 24 hours post application. In some embodiments, the transdermal delivery device is applied to provide a pharmacokinetic profile characterized by at least one of 1)-3) and/or at least one of 4)-8). For example, in some embodiments, the transdermal delivery device is applied for about 48 hours or more, and the pharmacokinetic profile can be characterized by at least 1), 2) and one or more of 4)-8). In some embodiments, the transdermal delivery device is applied for about 72 hours or more, and the pharmacokinetic profile can be characterized by all of 1)-8).

In some embodiments, the transdermal delivery device can include a reservoir layer comprising ketamine in an amount of about 5% to about 30%, preferably, about 5% to about 10% by weight of the reservoir layer, and applying the transdermal delivery device to the skin provides a pharmacokinetic profile in the human subject characterized by one or more of the following: i) ketamine concentration in plasma at 24 hours post application is about 10 ng/ml to about 1000 ng/mL; ii) ketamine concentration in plasma at 48 hours post application is about 15 ng/ml to about 100M ng/mL; iii) ketamine concentration in plasma at 72 hours post application is about 5 ng/ml to about 700 ng/mL; iv) a ratio of ketamine concentrations in plasma at 24 hours and 48 hours post application, C_{24 h}/C_{48 h}, is about 0.5 to about 1.1; v) a ratio of ketamine concentrations in plasma at 24 hours and 12 hours post application. C_{24 h}/C_{12 h}, is about 5 to about 15; vi) a ratio of ketamine concentrations in plasma at 18 hours and 12 hours post application, C_{18 h}/C_{12 h}, is about 5 to about 15; vii) a ratio of ketamine concentrations in plasma at 12 hours and 8 hours post application, C_{12 h}/C_{8 h}, is about 2 to about 6; and viii) ketamine concentration in plasma does not reach peak before 24 hours post application. In some embodiments, the transdermal delivery device is applied to provide a pharmacokinetic profile characterized by at least one of i)-iii) and/or at least one of iv)-viii). For example, in some embodiments, applying the transdermal delivery device provides at least pharmacokinetic characteristics i), v), vi) and vii) in the human subject. In some embodiments, applying the transdermal delivery device provides at least pharmacokinetic characteristics i), ii), iv) and viii) in the human subject. In some embodiments, the transdermal delivery device is applied for about 48 hours or more, and the pharmacokinetic profile can be characterized by at least i), ii) and one or more of iv)-viii). In some embodiments, the transdermal delivery device is applied for about 72 hours or more, and the pharmacokinetic profile can be characterized by all of i)-viii).

In some embodiments, the transdermal delivery device can include a reservoir layer comprising ketamine in an amount of about 5% to about 30%, preferably, about 5% to about 15% by weight of the reservoir layer, and applying the transdermal delivery device to the skin provides a pharmacokinetic profile in the human subject characterized by one or more of the following: i) ketamine concentration in plasma at 24 hours post application is about 10 ng/ml to about 1000 ng/mL; ii) ketamine concentration in plasma at 48 hours post application is about 15 ng/ml to about 1000 ng/mL; iii) ketamine concentration in plasma at 72 hours post application is about 5 ng/ml to about 1000 ng/mL; iv) a ratio of ketamine concentrations in plasma at 24 hours and 48 hours post application, C_{24 h}/C_{48 h}, is about 0.5 to about 1.1; v) a ratio of ketamine concentrations in plasma at 24 hours and 12 hours post application, C_{24 h}/C_{12 h}, is about 3 to about 8; vi) a ratio of ketamine concentrations in plasma at 18 hours and 12 hours post application, C_{18 h}/C_{12 h}, is about 3 to about 8; and vii) ketamine concentration in plasma does not reach peak before 24 hours post application. In some embodiments, the transdermal delivery device is applied to provide a pharmacokinetic profile characterized by at least one of i)-iii) and/or at least one of iv)-vii). For example, in some embodiments, applying the transdermal delivery device provides at least pharmacokinetic characteristics i), v), and vi) in the human subject. In some embodiments, applying the transdermal delivery device provides at least pharmacokinetic characteristics i), ii), iv) and vii) in the human subject. In some embodiments, the transdermal delivery device is applied for about 48 hours or more, and the pharmacokinetic profile can be characterized by at least i), ii) and one or more of iv)-vii). In some embodiments, the transdermal delivery device is applied for about 72 hours or more, and the pharmacokinetic profile can be characterized by all of i)-vii).

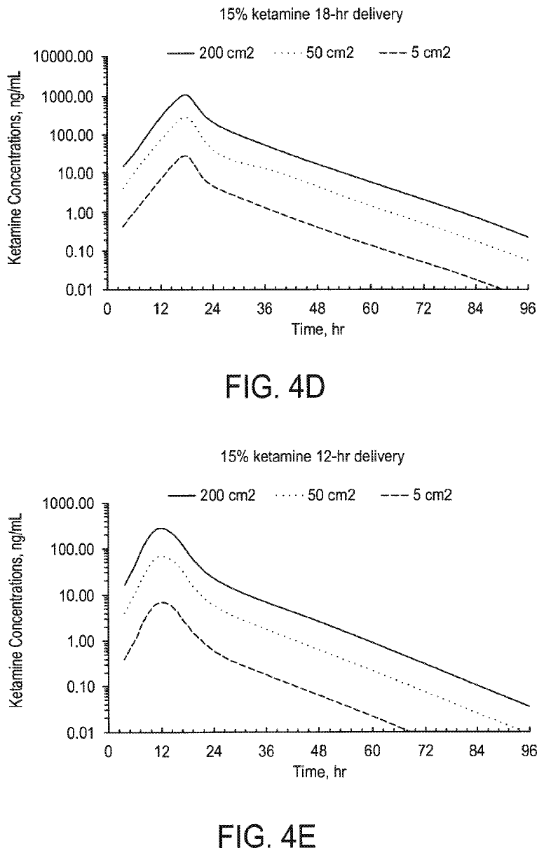

The pharmacokinetic profile described hereinabove can be achieved by selecting a proper transdermal delivery device, e.g., as described herein. As understood by those skilled in the art, plasma concentration of ketamine in a subject can depend on various factors, such as ketamine flux rate and size of the transdermal delivery device and the duration of application of the transdermal delivery device. The plasma concentrations of ketamine versus time can be calculated based on the reported pharmacokinetic parameters of ketamine in human. According to Fanta et. al., ketamine follows a three-compartment model with the parameters for a 70-kg human as follows: clearance=79.8 (liter/hour); V1=133 liter; and micro constants k12=0.174 hour⁻¹, k13=1.18 hour⁻¹, k21=0.124 hour⁻¹, k31=1.59 hour⁻¹, Fanta, et al., Eur. J. Clin. Pharmacol 71:441-447 (2015). Based on this model, Example 4 shows estimated plasma concentrations for a few exemplary transdermal delivery devices herein, which are sufficient to cover the desired concentrations described herein and can deliver a dose of ketamine, for example, ranging from about 5 mg to about 7 grams, see, e.g., FIG. 4A-4F.

Ketamine Analogs and Metabolites

Ketamine can be extensively metabolized with the major metabolites being norketamine and hydroxyketamines, dehydronorketamine, and hydroxynorketamines. See e.g., Zanos, P. et al., “NMDAR inhibition—independent antidepressant actions of ketamine metabolites,” Nature, S33(7604):481-6(2016), and U.S. Pat. No. 9,650,352. Further, deuterated ketamines (e.g., with at least one of the hydrogen atoms in ketamine being substituted by deuterium above its natural abundance, e.g., greater than 10% deuterium, or more than 90% deuterium) and deuterated norketamines (e.g., with at least one of the hydrogen atoms in norketamine being substituted by deuterium above its natural abundance, e.g., greater than 10% deuterium, or more than 90% deuterium) have been synthesized. See e.g., WO 2017/180589, US Pub. No. 2017/0355663, and U.S. Pat. No. 7,638,651.

In some embodiments, the present invention also provides a ketamine metabolite transdermal delivery device or gel formulation comprising one or more metabolites chosen from norketamine, hydroxyketamines, dehydronorketamine, and hydroxynorketamines. In some embodiments, the ketamine metabolite transdermal delivery device or gel formulation can be essentially the same as any of the respective ketamine transdermal delivery device or ketamine gel formulation described herein, except that the ketamine is substituted with one or more metabolites chosen from norketamine, hydroxyketamines, dehydronorketamine, and hydroxynorketamines. For example, in some embodiments, the ketamine metabolite transdermal delivery device can be a drug-in-adhesive or drug-in-reservoir patch. In some embodiments, the ketamine metabolite gel formulation can comprise the one or more ketamine metabolites, one or more permeation enhancers, and one or more gel-forming agents. Suitable permeation enhancers and gel-forming agents include those described herein. Other suitable ingredients such as adhesives, abuse deterrent agents, solvents, etc. are also described herein. In some embodiments, the active ingredient in the ketamine metabolite transdermal delivery device or gel formulation comprises, consists essentially of, or consists of hydroxynorketamine (“HNK”). In some embodiments, the HNK can be in the form of 2R,6R-isomer, 2S,6S-isomer, or any mixtures thereof. In some embodiments, the ketamine metabolite transdermal delivery device or gel formulation comprises a therapeutically effective amount of 2R,6R-hydroxynorketamine and is substantially free of 2S,6S-hydroxynorketamine (e.g., the ratio of the 2R,6R-isomer to the 2S,6S-isomer is more than 10:1, more than 20:1, or above). In some embodiments, the ketamine metabolite transdermal delivery device or gel formulation can also be used for the treatment of any of the diseases or disorders described herein, for example, depression (e.g., major depressive disorder, treatment-resistant depression, bipolar depression), anxiety, pain (e.g., neuropathic pain, complex regional pain syndrome (CRPS), chronic pain), etc.

In some embodiments, the present invention also provides a deuterated ketamine transdermal delivery device or gel formulation comprising one or more of the deuterated analogs chosen from deuterated ketamines and deuterated norketamines. In some embodiments, the deuterated ketamine transdermal delivery device or gel formulation is essentially the same as any of the respective ketamine transdermal delivery device or ketamine gel formulation described herein, except that the ketamine is substituted with one or more of the deuterated analogs chosen from deuterated ketamines and deuterated norketamines. For example, in some embodiments, the deuterated ketamine transdermal delivery device can be a drug-in-adhesive or drug-in-reservoir patch. In some embodiments, the deuterated ketamine gel formulation can comprise the one or more deuterated analogs, one or more permeation enhancers, and one or more gel-forming agents. Suitable permeation enhancers and gel-forming agents include those described herein. Other suitable ingredients such as adhesives, abuse deterrent agents, solvents, etc. are also described herein. In some embodiments, the active ingredient in the deuterated ketamine transdermal delivery device or gel formulation comprises, consists essentially of, or consists of a deuterated ketamine. In some embodiments, the deuterated ketamine transdermal delivery device or gel formulation can also be used for the treatment of any of the diseases or disorders described herein, for example, depression (e.g., major depressive disorder, treatment-resistant depression, bipolar depression), anxiety, pain (e.g., neuropathic pain, complex regional pain syndrome (CRPS), chronic pain), etc.

### Definitions

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

When applying a transdermal delivery device described herein to a skin of a subject, all of the adhesive surface can become in contact with the skin of the subject in theory. Thus, the area of the adhesive surface defines a contact area, i.e., a skin contact area, which is also referred to herein as “active surface area”. In a typical DIR patch design, a small portion of the skin contact surface may exceed the reservoir portion, such as the peripheral area of the adhesive surface, which is typically small. Thus, the reservoir portion area and the skin contact area are nearly identical. Unless otherwise obvious from context, the unit “/cm²” should be understood as per square centimeter of active surface area as defined herein. However, for any of the embodiments described herein where the recited value or range is based on the active surface area, alternative embodiments with the respective value or range based on the reservoir portion area are also provided herein.

As used herein, “coat weight” or “gel weight” of a drug-containing layer refers to the weight of the drug-containing layer (e.g., gel in the reservoir layer) per unit area of the active surface area of the transdermal drug delivery system.

As used herein, the term “cumulative drug permeated” refers to the total amount of drug permeated per square centimeter during a given period of time. Unless otherwise obvious from context, “cumulative drug permeated” at a given time (e.g., at 24 hours post administration) refers to the total amount of drug permeated per square centimeter from time 0 (i.e., time of administration) to the given time. Unless otherwise obvious from context, “cumulative drug permeated” refers to the arithmetic mean value measured and/or calculated according to the methods described herein. The term “mean value” as used herein, when not specified, also refers to arithmetic mean value, unless contradictory to common practice in the field.

As used herein, the term “flux” refers to the quantity of the drug permeated skin per unit area per unit time. As used herein, the term “average flux” refers to the total amount of drug permeated per square centimeter during a given period of time divided by the time duration. For example, an average flux from 12 hours to 24 hours post application would be calculated based on the total amount of drug permeated per square centimeter from 12 hours to 24 hours post application divided by 12 hours. As used herein, the term “steady state flux” refers to the flux observed during a period of time where the flux is substantially constant across the period (e.g., fluctuate within ±50% of the mean value observed for that period). Unless otherwise obvious from context, “flux”, “average flux”, or “steady state flux” refers to the arithmetic mean value measured and/or calculated according to the methods described herein. A typical unit of flux is milligram per square centimeter per hour.

Flux rate as referenced in this patent application can mean that measured by either in vivo or in vitro methods. One way to measure flux is to place the transdermal delivery device or formulation on a known skin area of a human volunteer and measure how much drug can permeate across skin within certain time constraints. In some embodiments, when specifically referenced as measured by in vitro method using human cadaver skin, the flux rate is measured in accordance with the method described in Example 3. Although an in vitro method uses human epidermal membrane obtained from a cadaver, rather than measure drug flux across the skin using human volunteers, it is generally accepted by those skilled in the art that results from a properly designed and executed in vitro test can be used to estimate or predict the results of an in vivo test with reasonable reliability.

The terms “skin flux characteristics” and “flux characteristics” are used interchangeably herein.

As used herein, the term “ketamine” refers to the base form, i.e., ketamine base, although it should be apparent to those skilled in the art that upon mixing with other ingredient(s), ketamine can become protonated. To be clear, in embodiments herein, the transdermal delivery device or the ketamine gel formulation comprising ketamine in a recited amount should be understood such that the total amount of ketamine, regardless of its protonation state, is present in the recited amount when expressed as the equivalent amount of ketamine base. For example, in any of the embodiments described herein, the transdermal delivery device or the ketamine gel formulation comprising ketamine in a recited amount can be prepared by a method comprising mixing directly or indirectly ketamine base in the recited amount with other ingredients/components, see e.g., Example 1. Further, ketamine concentration, flux, and the alike as referenced herein should be understood as those measured and/or calculated in accordance with the methods described herein, with the final numeric value expressed as the equivalent value for ketamine base. For example, a ketamine flux of 1 mg/day/cm² should be understood as the total amount of ketamine permeated per day per cm², regardless of its protonation state, measured and/or calculated to be 1 mg when expressed as the equivalent value of ketamine base.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

The term “therapeutically effective amount,” as used herein, refers to that amount of a therapeutic agent (e.g., ketamine) sufficient to result in amelioration of one or more symptoms of a disorder or condition (e.g., pain, depression), or prevent appearance or advancement of a disorder or condition, or cause regression of or cure from the disorder or condition.

The term “subject” (alternatively referred to herein as “patient”) as used herein, refers to an animal, preferably a mammal, most preferably a human, who has been the object of treatment, observation or experiment.

As used herein, applying or administering the transdermal delivery device herein should be understood as in accordance with how such transdermal delivery device is normally applied or administered, e.g., to the skin of a human subject.

### Alternative Embodiments

1. A transdermal delivery device for administering ketamine, comprising:

a backing layer,

a reservoir layer comprising ketamine in an amount of about 2% to about 30% by weight of the reservoir layer, and

an adhesive layer defining an active surface area,

wherein the transdermal delivery device is configured to provide one or more of the following skin flux characteristics when tested in vitro using human cadaver skin:
(a) a cumulative ketamine permeated of about 0.04 mg/cm² to about 3 mg/cm² at 12 hours post administration based on the active surface area;
(b) a cumulative ketamine permeated of about 0.8 mg/cm² to about 20 mg/cm² at 24 hours post administration based on the active surface area;
(c) a cumulative ketamine permeated of about 2.5 mg/cm² to about 65 mg/cm² at 48 hours post administration based on the active surface area;
(d) a cumulative ketamine permeated of about 3 mg/cm² to about 85 mg/cm² at 72 hours post administration based on the active surface area;
(e) an average flux of ketamine of about 0.005 mg/cm²*h to about 0.4 mg/cm²*h from 4 hours to 12 hours post administration;
(f) an average flux of ketamine of about 0.06 mg/cm²*h to about 1.4 mg/cm²*h from 12 hours to 18 hours post administration;
(g) an average flux of ketamine of about 0.06 mg/cm²*h to about 1.4 mg/cm²*h from 12 hours to 24 hours post administration;
(h) a steady state flux of ketamine of about 0.06 mg/cm²*h to about 1.8 mg/cm²*h;
(i) an average flux of ketamine of about 0.08 mg/cm²*h to about 1.8 mg/cm²*h from 24 hours to 48 hours post administration; and
(j) an average flux of ketamine of about 0.03 mg/cm²*h to about 0.9 mg/cm²*h from 48 hours to 72 hours post administration.
2. The transdermal delivery device of embodiment 1, configured to provide one or more of skin flux characteristics of (a) to (d) and one or more of skin flux characteristics of (e) to (j), when tested in vitro using human cadaver skin.
3. The transdermal delivery device of embodiment 1 or 2, wherein the ketamine is present in an amount of about 2% to about 5% by weight of the reservoir layer, wherein the transdermal delivery device is configured to provide one or more of the following skin flux characteristics when tested in vitro using human cadaver skin:
   1) a cumulative ketamine permeated of about 0.04 mg/cm² to about 0.2 mg/cm² at 12 hours post administration based on the active surface area;
   2) a cumulative ketamine permeated of about 0.8 mg/cm² to about 3.5 mg/cm² at 24 hours post administration based on the active surface area;
   3) a cumulative ketamine permeated of about 2.5 mg/cm² to about 11 mg/cm² at 48 hours post administration based on the active surface area;
   4) a cumulative ketamine permeated of about 3 mg/cm² to about 15 mg/cm² at 72 hours post administration based on the active surface area;
   5) an average flux of ketamine of about 0.005 mg/cm²*h to about 0.03 mg/cm²*h from 4 hours to 12 hours post administration;
   6) an average flux of ketamine of about 0.06 mg/cm²*h to about 0.26 mg/cm²*h from 12 hours to 18 hours post administration;
   7) an average flux of ketamine of about 0.06 mg/cm²*h to about 0.26 mg/cm²*h from 12 hours to 24 hours post administration;
   8) a steady state flux of ketamine of about 0.06 mg/cm²*h to about 0.31 mg/cm²*h;
   9) an average flux of ketamine of about 0.07 mg/cm²*h to about 0.31 mg/cm²*h from 24 hours to 48 hours post administration; and
   10) an average flux of ketamine of about 0.03 mg/cm²*h to about 0.12 mg/cm²*h from 48 hours to 72 hours post administration.
4. The transdermal delivery device of embodiment 3, configured to provide one or more of skin flux characteristics of 1) to 4) and one or more of skin flux characteristics of 5) to 10), when tested in vitro using human cadaver skin.
5. The transdermal delivery device of embodiment 1 or 2, wherein the ketamine is present in an amount of about 5% to about 10% by weight of the reservoir layer, wherein the transdermal delivery device is configured to provide one or more of the following skin flux characteristics when tested in vitro using human cadaver skin:
   1) a cumulative ketamine permeated of about 0.1 mg/cm² to about 0.4 mg/cm² at 12 hours post administration based on the active surface area;
   2) a cumulative ketamine permeated of about 2 mg/cm² to about 7 mg/cm² at 24 hours post administration based on the active surface area;
   3) a cumulative ketamine permeated of about 6 mg/cm² to about 25 mg/cm² at 48 hours post administration based on the active surface area;
   4) a cumulative ketamine permeated of about 8 mg/cm² to about 30 mg/cm² at 72 hours post administration based on the active surface area;
   5) an average flux of ketamine of about 0.013 mg/cm²*h to about 0.05 mg/cm²*h from 4 hours to 12 hours post administration;
   6) an average flux of ketamine of about 0.15 mg/cm²*h to about 0.6 mg/cm²*h from 12 hours to 18 hours post administration;
   7) an average flux of ketamine of about 0.15 mg/cm²*h to about 0.6 mg/cm²*h from 12 hours to 24 hours post administration;
   8) a steady state flux of ketamine of about 0.15 mg/cm²*h to about 0.7 mg/cm²*h;
   9) an average flux of ketamine of about 0.19 mg/cm²*h to about 0.7 mg/cm²*h from 24 hours to 48 hours post administration; and
   10) an average flux of ketamine of about 0.07 mg/cm²*h to about 0.3 mg/cm²*h from 48 hours to 72 hours post administration.
6. The transdermal delivery device of embodiment 5, configured to provide one or more of skin flux characteristics of 1) to 4) and one or more of skin flux characteristics of 5) to 10).
7. The transdermal delivery device of embodiment 1 or 2, wherein the ketamine is present in an amount of about 10% to about 20% by weight of the reservoir layer, wherein the transdermal delivery device is configured to provide one or more of the following skin flux characteristics when tested in vitro using human cadaver skin:
   1) a cumulative ketamine permeated of about 0.2 mg/cm² to about 0.4 mg/cm² at 12 hours post administration based on the active surface area;
   2) a cumulative ketamine permeated of about 4 mg/cm² to about 7 mg/cm² at 24 hours post administration based on the active surface area;
   3) a cumulative ketamine permeated of about 13 mg/cm² to about 25 mg/cm² at 48 hours post administration based on the active surface area;
   4) a cumulative ketamine permeated of about 17 mg/cm² to about 30 mg/cm² at 72 hours post administration based on the active surface area;
   5) an average flux of ketamine of about 0.02 mg/cm²*h to about 0.05 mg/cm²*h from 4 hours to 12 hours post administration;
   6) an average flux of ketamine of about 0.3 mg/cm²*h to about 0.6 mg/cm²*h from 12 hours to 18 hours post administration;
   7) an average flux of ketamine of about 0.3 mg/cm²*h to about 0.6 mg/cm²*h from 12 hours to 24 hours post administration;
   8) a steady state flux of ketamine of about 0.3 mg/cm²*h to about 0.7 mg/cm²*h;
   9) an average flux of ketamine of about 0.35 mg/cm²*h to about 0.7 mg/cm²*h from 24 hours to 48 hours post administration; and
   10) an average flux of ketamine of about 0.15 mg/cm²*h to about 0.3 mg/cm²*h from 48 hours to 72 hours post administration.
8. The transdermal delivery device of embodiment 7, configured to provide one or more of skin flux characteristics of 1) to 4) and one or more of skin flux characteristics of 5) to 10), when tested in vitro using human cadaver skin.
9. The transdermal delivery device of embodiment 7 or 8, configured to provide skin flux characteristics comprising 1), 2), 5), 6), and 7), when tested in vitro using human cadaver skin.
10. The transdermal delivery device of any one of embodiments 7-9, configured to provide skin flux characteristics comprising 1), 2), and 8), when tested in vitro using human cadaver skin.
11. The transdermal delivery device of any one of embodiments 7-10, configured to provide skin flux characteristics of 3) and/or 9).
12. The transdermal delivery device of any one of embodiments 7-11, configured to provide skin flux characteristics of 4) and/or 10).
13. The transdermal delivery device of any one of embodiments 7-12, wherein the ketamine is present in an amount of about 10% by weight of the reservoir layer.
14. The transdermal delivery device of embodiment 1 or 2, wherein the ketamine is present in an amount of about 15% to about 20% by weight of the reservoir layer, wherein the transdermal delivery device is configured to provide one or more of the following skin flux characteristics when tested in vitro using human cadaver skin:
   1) a cumulative ketamine permeated of about 0.9 mg/cm² to about 1.5 mg/cm² at 12 hours post administration based on the active surface area;
   2) a cumulative ketamine permeated of about 6 mg/cm² to about 10 mg/cm² at 24 hours post administration based on the active surface area;
   3) a cumulative ketamine permeated of about 19 mg/cm² to about 35 mg/cm² at 48 hours post administration based on the active surface area;
   4) a cumulative ketamine permeated of about 25 mg/cm² to about 45 mg/cm² at 72 hours post administration based on the active surface area;
   5) an average flux of ketamine of about 0.1 mg/cm²*h to about 0.2 mg/cm²*h from 4 hours to 12 hours post administration;
   6) an average flux of ketamine of about 0.4 mg/cm²*h to about 0.7 mg/cm²*h from 12 hours to 18 hours post administration;
   7) an average flux of ketamine of about 0.4 mg/cm²*h to about 0.7 mg/cm²*h from 12 hours to 24 hours post administration;
   8) a steady state flux of ketamine of about 0.4 mg/cm²*h to about 0.9 mg/cm²*h;
   9) an average flux of ketamine of about 0.5 mg/cm²*h to about 0.9 mg/cm²*h from 24 hours to 48 hours post administration; and
   10) an average flux of ketamine of about 0.25 mg/cm²*h to about 0.45 mg/cm²*h from 48 hours to 72 hours post administration.
15. The transdermal delivery device of embodiment 14, configured to provide one or more of skin flux characteristics of 1) to 4) and one or more of skin flux characteristics of 5) to 10), when tested in vitro using human cadaver skin.
16. The transdermal delivery device of embodiment 14 or 15, configured to provide skin flux characteristics comprising 1), 2), 5), 6), and 7), when tested in vitro using human cadaver skin.
17. The transdermal delivery device of embodiment 14 or 15, configured to provide skin flux characteristics comprising 1), 2), and 8), when tested in vitro using human cadaver skin.
18. The transdermal delivery device of any one of embodiments 14-17, configured to provide skin flux characteristics of 3) and/or 9).
19. The transdermal delivery device of any one of embodiments 14-18, configured to provide skin flux characteristics of 4) and/or 10).
20. The transdermal delivery device of any one of embodiments 14-19, wherein the ketamine is present in an amount of about 15% by weight of the reservoir layer.
21. The transdermal delivery device of any one of embodiments 1-20, wherein the active surface area is about 5 cm² to about 300 cm².
22. The transdermal delivery device of any one of embodiments 1-21, wherein the active surface area is about 10 cm² to about 100 cm².
23. The transdermal delivery device of any one of embodiments 1-22, comprising a rate-controlling membrane, wherein the rate-controlling membrane is in between the reservoir layer and the adhesive layer.
24. The transdermal delivery device of embodiment 23, wherein the rate-controlling membrane is a microporous membrane comprising a polypropylene film (e.g., Celgard 2400, Celgard 2500), a poly ethylene vinyl acetate film (e.g., CoTran 9702), or a combination thereof.
25. The transdermal delivery device of any one of embodiments 1-24, wherein the adhesive layer comprises a pressure sensitive adhesive.
26. The transdermal delivery device of embodiment 25, wherein the adhesive layer is about 1.5 mils to about 10 mils (e.g., about 1.5 mils to about 2 mils) thick.
27. The transdermal delivery device of embodiment 25 or 26, wherein the pressure sensitive adhesive comprises a polyisobutylene adhesive, a silicone polymer adhesive, an acrylate copolymer adhesive (e.g., a poly acrylate vinyl acetate copolymer, such as Duro-Tak 87-2287, Duro-Tak 87-4098, Duro-Tak 87-4287, or Duro-Tak 87-2516), or a combination thereof.
28. The transdermal delivery device of any one of embodiments 1-27, wherein the reservoir layer comprises a permeation enhancer.
29. The transdermal delivery device of embodiment 28, wherein the permeation enhancer is one or more compounds chosen from sulfoxides, alcohols, alkanols, glycols, and surfactants.
30. The transdermal delivery device of embodiment 28, wherein the permeation enhancer is one or more compounds chosen from dimethyl sulfoxide (DMSO), oleic alcohol, oleic acid, levulinic acid, propylene glycol, dipropylene glycol, ethanol, and surfactants such as Tween 80.
31. The transdermal delivery device of any one of embodiments 1-30, wherein the reservoir layer comprises a solvent.
32. The transdermal delivery device of embodiment 31, wherein the solvent comprises ethanol, water, propylene glycol, acetone, isopropyl alcohol, butylene glycol, dimethyl sulfoxide (DMSO), dimethyl acetamide (DMA), or a combination thereof.
33. The transdermal delivery device of any one of embodiments 1-32, wherein the reservoir layer comprises a gel-forming agent in a gel-forming amount.
34. The transdermal delivery device of embodiment 33, wherein the gel-forming agent comprises a hydroxypropylcellulose (e.g., Klucel HF Pharm), hydroxypropyl methyl cellulose (HPMC), polyvinyl pyrrolidone (PVP such as BASF's Kollidon), polyacrylic acid (such as Carbopol), sodium CMC (carboxyl methyl cellulose), or a combination thereof.
35. The transdermal delivery device of any one of embodiments 1-34, further comprising an abuse deterrent agent selected from aversive agents such as capsaicin, apomorphine, denatonium, sodium laurel sulfate, niacin and combinations thereof.
36. The transdermal delivery device of embodiment 35, wherein the abuse deterrent agent is present in the reservoir layer, the adhesive layer, and/or in a separate layer.
37. The transdermal delivery device of any one of embodiments 1-36, wherein the reservoir layer has a coat weight of about 0.15 g/cm² to about 0.24 g/cm² active surface area.
38. The transdermal delivery device of any one of embodiments 1-36, wherein the reservoir layer has a thickness of about 2 mm to about 3.5 mm.
39. The transdermal delivery device of any one of embodiments 1-36, wherein the reservoir layer comprises an amount of ketamine sufficient to provide about 0.1 mg/day/cm² to about 30 mg/day/cm² of ketamine over a period of time selected from about 8 hours, about 12 hours, about 18 hours, about 24 hours, about 2 days, about 3 days, about 4 days, about 5 days, about 6 days, and about 7 days.
40. The transdermal delivery device of any one of embodiments 1-36, wherein the reservoir layer comprises an amount of ketamine sufficient to provide about 1 mg/day/cm² to about 5 mg/day/cm² or about 2 mg/day/cm² to about 10 mg/day/cm² of ketamine over a period of time selected from about 24 hours, about 2 days, about 3 days, about 4 days, about 5 days, about 6 days, and about 7 days.
41. A method of administering ketamine through the skin of a human subject in need thereof, the method comprising applying the transdermal delivery device of any one of embodiments 1-40 to the human subject such that substantially all of the active surface area of the transdermal delivery device is in contact with the skin of the human subject.
42. The method of embodiment 41, wherein the human subject is characterized as having a major depressive disorder, and the method delivers about 1 mg/day/cm² to about 5 mg/day/cm² of ketamine to the human subject.
43. The method of embodiment 41, wherein the human subject is characterized as having pain, and the method delivers about 2 mg/day/cm² to about 10 mg/day/cm² of ketamine to the human subject.
44. A method of administering ketamine through the skin of a human subject in need thereof, the method comprising applying a transdermal delivery device to the skin of the human subject, wherein the transdermal delivery device comprises a backing layer; a reservoir layer comprising ketamine in an amount of about 2% to about 30% by weight of the reservoir layer, and an adhesive layer defining an active surface area, wherein the active surface area is about 5 cm² to about 200 cm², and
   wherein the transdermal delivery device is applied such that substantially all of the active surface area of the transdermal delivery device is in contact with the skin of the human subject, wherein applying the transdermal delivery device provides one or more of the following pharmacokinetic characteristics in the human subject:
   1) ketamine concentration in plasma at 24 hours post application is about 5 ng/ml to about 1000 ng/mL;
   2) ketamine concentration in plasma at 48 hours post application is about 6 ng/ml to about 1000 ng/mL;
   3) ketamine concentration in plasma at 72 hours post application is about 3 ng/ml to about 1000 ng/mL;
   4) a ratio of ketamine concentrations in plasma at 24 hours and 48 hours post application, C_{24 h}/C_{48 h}, is about 0.5 to about 1.5;
   5) a ratio of ketamine concentrations in plasma at 24 hours and 12 hours post application, C_{24 h}/C_{12 h}, is about 3 to about 20;
   6) a ratio of ketamine concentrations in plasma at 18 hours and 12 hours post application, C_{18 h}/C_{12 h}, is about 3 to about 20;
   7) a ratio of ketamine concentrations in plasma at 12 hours and 8 hours post application, C_{12 h}/C_{8 h}, is about 2 to about 10; and
   8) ketamine concentration in plasma does not reach peak before 24 hours post application.
45. The method of embodiment 44, wherein the reservoir layer comprising ketamine in an amount of about 5% to about 10% by weight of the reservoir layer, and
   wherein applying the transdermal delivery device provides one or more of the following pharmacokinetic characteristics in the human subject:
   i. ketamine concentration in plasma at 24 hours post application is about 10 ng/ml to about 1000 ng/mL;
   ii. ketamine concentration in plasma at 48 hours post application is about 15 ng/ml to about 1000 ng/mL;
   iii. ketamine concentration in plasma at 72 hours post application is about 5 ng/ml to about 700 ng/mL;
   iv. a ratio of ketamine concentrations in plasma at 24 hours and 48 hours post application, C_{24 h}/C_{48 h}, is about 0.5 to about 1.1;
   v. a ratio of ketamine concentrations in plasma at 24 hours and 12 hours post application, C_{24 h}/C_{12 h}, is about 5 to about 15;
   vi. a ratio of ketamine concentrations in plasma at 18 hours and 12 hours post application, C_{18 h}/C_{12 h}, is about 5 to about 15;
   vii. a ratio of ketamine concentrations in plasma at 12 hours and 8 hours post application, C_{12 h}/C_{8 h}, is about 2 to about 6; and
   viii. ketamine concentration in plasma does not reach peak before 24 hours post application.
46. The method of embodiment 45, wherein applying the transdermal delivery device provides one or more of pharmacokinetic characteristics (i)-(iii) and one or more of pharmacokinetic characteristics (iv)-(viii) in the human subject.
47. The method of embodiment 45, wherein applying the transdermal delivery device provides at least pharmacokinetic characteristics (i), (v), (vi) and (vii) in the human subject.
48. The method of embodiment 45, wherein applying the transdermal delivery device provides at least pharmacokinetic characteristics (i), (ii), (iv) and (viii) in the human subject.
49. The method of embodiment 44, wherein the reservoir layer comprising ketamine in an amount of about 5% to about 15% by weight of the reservoir layer, and
   wherein applying the transdermal delivery device provides one or more of the following pharmacokinetic characteristics in the human subject:
   i. ketamine concentration in plasma at 24 hours post application is about 10 ng/ml to about 1000 ng/mL;
   ii. ketamine concentration in plasma at 48 hours post application is about 15 ng/ml to about 1000 ng/mL;
   iii. ketamine concentration in plasma at 72 hours post application is about 5 ng/ml to about 1000 ng/mL;
   iv. a ratio of ketamine concentrations in plasma at 24 hours and 48 hours post application, C_{24 h}/C_{48 h}, is about 0.5 to about 1.1;
   v. a ratio of ketamine concentrations in plasma at 24 hours and 12 hours post application, C_{24 h}/C_{12 h}, is about 3 to about 8;
   vi. a ratio of ketamine concentrations in plasma at 18 hours and 12 hours post application, C_{18 h}/C_{12 h}, is about 3 to about 8; and
   vii. ketamine concentration in plasma does not reach peak before 24 hours post application.
50. The method of embodiment 49, wherein applying the transdermal delivery device provides one or more of pharmacokinetic characteristics (i)-(iii) and one or more of pharmacokinetic characteristics (iv)-(vii) in the human subject.
51. The method of embodiment 49, wherein applying the transdermal delivery device provides at least pharmacokinetic characteristics (i), (v), and (vi) in the human subject.
52. The method of embodiment 49, wherein applying the transdermal delivery device provides at least pharmacokinetic characteristics (i), (ii), (iv) and (vii) in the human subject.
53. The method of any one of embodiments 44-52, wherein the human subject is characterized as having a major depressive disorder.
54. The method of any one of embodiments 44-52, wherein the human subject is characterized as having pain.
55. A ketamine gel formulation comprising ketamine in the amount of about 2% to about 30% by weight, a solvent in the amount of about 40% to about 75% by weight, a permeation enhancer of about 5% to about 25% by weight, and a gel-forming agent in a gel-forming amount.
56. The ketamine gel of embodiment 55, wherein the permeation enhancer comprises one or more compounds chosen from sulfoxides, alcohols, alkanols, glycols, and surfactants.
57. The ketamine gel of embodiment 55, wherein the permeation enhancer comprises one or more compounds chosen from dimethyl sulfoxide (DMSO), oleic alcohol, oleic acid, levulinic acid, propylene glycol, dipropylene glycol, ethanol, and surfactants such as Tween 80.
58. The ketamine gel of any one of embodiments 55-57, wherein the permeation enhancer comprises one to three of the following: (a) levulinic acid in the amount of about 0.1% to about 15% by weight; (b) oleic acid in the amount of about 0.1% to about 10% by weight; (c) oleic alcohol in the amount of about 0.1% to about 10% by weight; (d) DMSO in the amount of about 0.1% to about 10% by weight; and (e) dipropylene glycol in the amount of about 0.1% to about 15% by weight.
59. The ketamine gel of any one of embodiments 55-58, wherein the permeation enhancer comprises levulinic acid in the amount of about 5% to about 15% by weight and oleic alcohol in the amount of about 1% to about 8% by weight.
60. The ketamine gel of any one of embodiments 55-59, wherein the permeation enhancer comprises DMSO in the amount of about 0.1% to about 10% by weight.
61. The ketamine gel of any one of embodiments 55-58, wherein the permeation enhancer comprises levulinic acid in the amount of about 5% to about 15% by weight and oleic acid in the amount of about 1% to about 8% by weight.
62. The ketamine gel of any one of embodiments 55-58, wherein the permeation enhancer the permeation enhancer comprises levulinic acid in the amount of about 1% to about 10% by weight and dipropylene glycol in the amount of about 5% to about 15% by weight.
63. The ketamine gel of any one of embodiments 55-62, wherein the solvent comprises ethanol, water, propylene glycol, acetone, isopropyl alcohol, butylene glycol, dimethyl sulfoxide (DMSO), dimethyl acetamide (DMA), or a combination thereof.
64. The ketamine gel of any one of embodiments 55-63, wherein the gel-forming agent comprises a hydroxypropylcellulose, hydroxypropyl methyl cellulose (HPMC), polyvinyl pyrrolidone (PVP such as BASF's Kollidon), polyacrylic acid (such as Carbopol), sodium CMC (carboxyl methyl cellulose), or a combination thereof.
65. The ketamine gel of any one of embodiments 55-64, further comprising an abuse deterrent agent selected from aversive agents such as capsaicin, apomorphine, denatonium, sodium laurel sulfate, niacin and combinations thereof.
66. The ketamine gel of any one of embodiments 55-65, characterized by a viscosity of about 15,000 cP to about 45,000 cP.
67. A transdermal delivery device for administering ketamine, comprising:
   a backing layer (e.g., polyester such as Scotchpak 9736, polyurethane film such as Scotchpak 9701, polyethylene film such as CoTran 9720),
   a reservoir layer comprising the ketamine gel of any one of embodiments 55-66, an adhesive layer defining an active surface area, and
   a release liner (e.g., 3M's ScotchPak 9744).
68. The transdermal delivery device of embodiment 67, wherein the adhesive layer is a separate layer configured to contact the skin of a human subject and the reservoir layer is in between the adhesive layer and the backing layer.
69. The transdermal delivery device of embodiment 67 or 68, wherein the adhesive layer comprises a pressure sensitive adhesive.
70. The transdermal delivery device of any one of embodiments 67-69, wherein the adhesive layer is about 1.5 mils to about 10 mils (e.g., about 1.5 mils to about 2 mils) thick.
71. The transdermal delivery device of embodiment 69 or 70, wherein the pressure sensitive adhesive comprises a polyisobutylene adhesive, a silicone polymer adhesive, an acrylate copolymer adhesive, or a combination thereof.
72. The transdermal delivery device of any one of embodiments 67-71, further comprising a rate-controlling membrane, wherein the rate-controlling membrane is in between the reservoir layer and the adhesive layer.
73. The transdermal delivery device of embodiment 72, wherein the rate-controlling membrane is a microporous membrane comprising a polypropylene film, a poly ethylene vinyl acetate film, or a combination thereof.
74. The transdermal delivery device of any one of embodiments 67-73, wherein the active surface area is about 5 cm² to about 300 cm².
75. The transdermal delivery device of embodiment 74, wherein the active surface area is about 10 cm² to about 100 cm².
76. The transdermal delivery device of any one of embodiments 67-75, further comprising an abuse deterrent layer, wherein the abuse deterrent layer comprises an abuse deterrent agent selected from aversive agents such as capsaicin, apomorphine, denatonium, sodium laurel sulfate, niacin, and combinations thereof.
77. The transdermal delivery device of any one of embodiments 67-76, wherein the reservoir layer comprises an amount of ketamine sufficient to provide about 0.1 mg/day/cm² to about 30 mg/day/cm² of ketamine over a period of time selected from about 8 hours, about 12 hours, about 18 hours, about 24 hours, about 2 days, about 3 days, about 4 days, about 5 days, about 6 days, and about 7 days.
78. The transdermal delivery device of any one of embodiments 67-77, wherein the ketamine is present in the amount of about 5% to about 10% by weight of the reservoir layer.
79. The transdermal delivery device of any one of embodiments 67-78, wherein the reservoir layer comprises an amount of ketamine sufficient to provide about 1 mg/day/cm² to about 5 mg/day/cm² or about 2 mg/day/cm² to about 10 mg/day/cm² of ketamine over a period of time selected from about 24 hours, about 2 days, about 3 days, about 4 days, about 5 days, about 6 days, and about 7 days.
80. The transdermal delivery device of any one of embodiments 67-79, which is storage stable at room temperature.
81. The transdermal delivery device of embodiment 80, wherein upon storage at 25° C. for 5 months after preparation, the transdermal delivery device provides one or more of the following skin flux characteristics when tested in vitro using human cadaver skin:
   (a) a cumulative ketamine permeated at 24 hours post administration of about 50% to about 250% of that observed for the same transdermal delivery device stored at 25° C. for 1 week after preparation;
   (b) a cumulative ketamine permeated at 48 hours post administration of about 60% to about 160% of that observed for the same transdermal delivery device stored at 25° C. for 1 week after preparation; and
   (c) a cumulative ketamine permeated at 72 hours post administration of about 75% to about 160% of that observed for the same transdermal delivery device stored at 25° C. for 1 week after preparation.
82. The transdermal delivery device of any one of embodiments 1-40 and 67-81 or the ketamine gel of any one of embodiments 55-66, which is free of a crystallization inhibitor selected from polyvinyl pyrrolidone-co-vinyl acetate and polymethacrylate.
83. The transdermal delivery device of any one of embodiments 1-40 and 67-81 or the ketamine gel of any one of embodiments 55-66, which is free of a crystallization inhibitor.
84. The transdermal delivery device of any one of embodiments 1-40 and 67-83 or the ketamine gel of any one of embodiments 55-66, wherein the ketamine exists in the form of a substantially pure S-enantiomer or a substantially pure R-enantiomer.
85. The transdermal delivery device of any one of embodiments 1-40 and 67-83 or the ketamine gel of any one of embodiments 55-66, wherein the ketamine exists in a racemic form.
86. A method of treating major depressive disorders in a subject in need thereof, comprising applying to the subject the transdermal delivery device of any one of embodiments 1-40 and 67-85 or the ketamine gel of any one of embodiments 55-66.
87. A method of treating pain in a subject in need thereof, comprising applying to the subject the transdermal delivery device of any one of embodiments 1-40 and 67-85 or the ketamine gel of any one of embodiments 55-66.
88. The method of embodiment 86 or 87, wherein the subject is a human subject.
89. The transdermal delivery device of any one of embodiments 1-40 and 67-85 or the ketamine gel of any one of embodiments 55-66, further comprising an antioxidant (e.g., one or more chosen from butylated hydroxyanisole (BHA), butylhydroxy toluene (BHT), tert-Butylhydroquinone, ascorbic acid, and tocopherols).
90. A method of treating a disease or disorder in a subject in need thereof, comprising applying to the subject the transdermal delivery device of any one of embodiments 1-40 and 67-85 or the ketamine gel of any one of embodiments 55-66, wherein the disease or disorder is one or more chosen from pain (e.g., neuropathic pain, complex regional pain syndrome (CRPS), chronic pain), depression (major depressive disorder, treatment-resistant depression, bipolar depression), restless legs syndrome, a condition associated with spinal cord injury (e.g., autonomic dysreflexia, immune suppression, chronic central neuropathic pain suffering from spinal cord injury, leukocyte apoptosis, splenic atrophy, leucopenia, or combinations thereof), anxiety, bipolar disorder (e.g., childhood-onset bipolar disorder, bipolar depression), stress-induced disorder (e.g., stress-induced affective disorder, stress-induced psychopathology), post-traumatic stress disorder, Alzheimer's dementia, amyotrophic lateral sclerosis, and suicidality.

### EXAMPLES

### Example 1. Ketamine Gel Formulation

The gel formulation was prepared by mixing (blending) the solvents, permeation enhancers, ketamine base, and the gelling agent listed in Table 4 below. The gel formulation can be used for preparing the ketamine DIR patches.

TABLE 4
Composition of ketamine gel

| Ingredient | wt % | wt % |
|---|---|---|
| Ketamine base | 15% | 10% |
| Alcohol USP | 60% | 67% |
| Oleic alcohol | 5% | 5% |
| Levulinic acid | 9% | 7% |
| Propylene glycol | 9% | 9% |
| Klucel HF (thickener) | 2% | 2% |
| TOTAL | 100% | 100% |

### Example 2. Ketamine DIR Patch—Preparation of Reservoir Patch Formulations

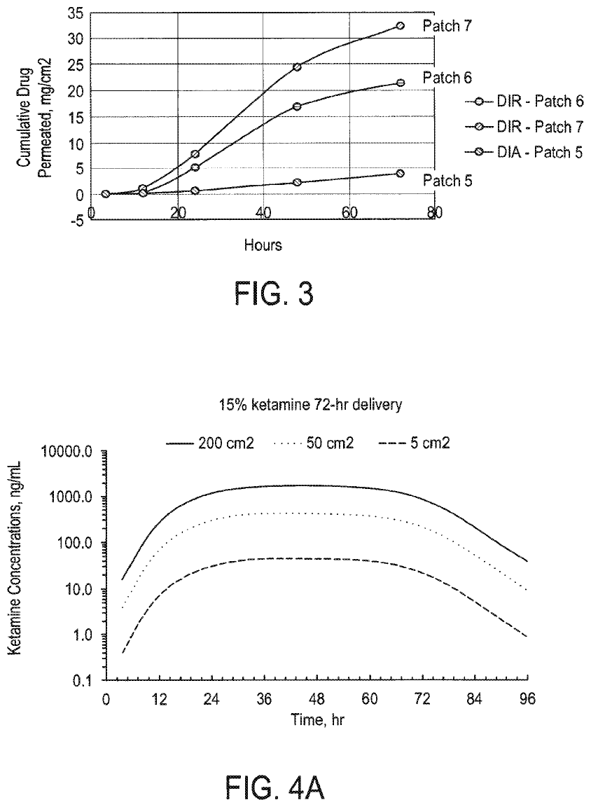

The reservoir patch devices include four main components: an impermeable backing, a drug reservoir which contains the drug dissolved in a gel vehicle, a microporous membrane, and an adhesive (FIG. 1). Reservoir transdermal patches are commercially available and the manufacturing methods well established. An exemplary process is shown in FIG. 2 and explained below.

A polypropylene membrane with 38% porosity (Celgard 2400) was used as the rate-controlling membrane. A heat-sealable polyester film (3M's 9730) was used as the backing layer. Pressure-sensitive adhesives were cast onto the release liner (e.g., No. 1022, 3M Company), and dried. A layer of Celgard 2400 was placed on top of the pressure-sensitive adhesive. The backing film was laid on top of the membrane, and heat sealing of the rim of a 1.77 cm² circle proceeds using a die compressed for 10 seconds at 70° C. to form an empty pouch reservoir between the Celgard and backing layer.

The gel formulations (10 and 15% drug, in alcoholic gel solution, prepared according to Example 1 with composition given in Table 4) were loaded into the patch reservoir by inserting a hypodermic syringe into the orifice leading into the reservoir chamber, filling it with 0.5 mL of the ketamine gel solution and then sealing the orifice. Patches were stored for one week prior to testing to allow the reservoir components to equilibrate with the pressure-sensitive adhesive.

### Example 3. In Vitro Flux Studies

The reservoir patches (DIR, drug-in-reservoir) prepared were placed on the human cadaver skin preparation (commercially available, e.g., through New York Fire Fighters Skin Bank, New York, N.Y.), and then mounted in the Franz cell for the skin flux study. Skin permeation studies were performed for the patch formulations using Franz diffusion cells kept at 37° C. for the duration of the experiment. The receptor medium was phosphate buffered saline at pH 7.4, the receptor volume 12 ml and the permeation area 1.77 cm². Human cadaver skin was used and all tests were performed in triplicate and the results below show the arithmetic mean value. The patch sample was placed at the donor site of the skin diffusion cells and the experiment was initiated with the receptor medium being continuously mixed. Samples of the receptor phase were obtained at 2, 4, 8, 12, 24, 48 and 72 h and ketamine concentrations were determined using HPLC method.

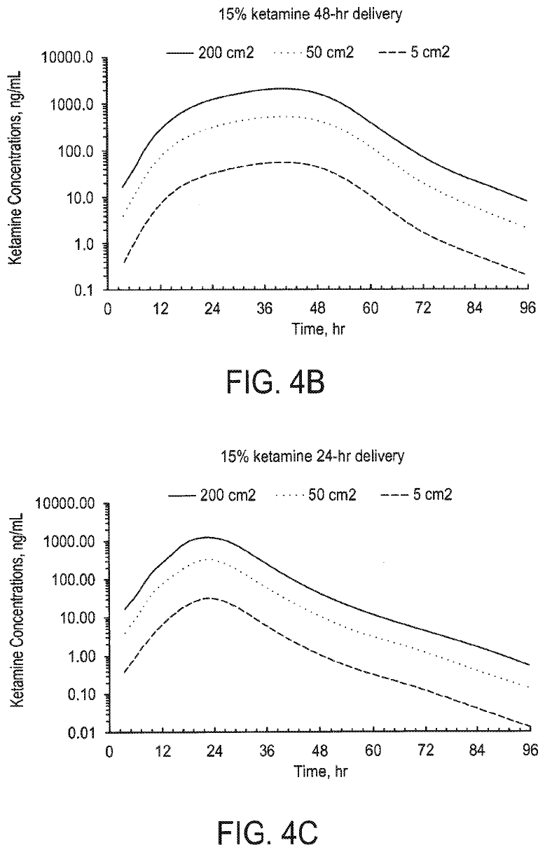

Patches 6 and 7 includes in the reservoir layer the 10% and 15% gel formulation in Example 1, respectively. Results of the skin flux study are shown as plots in FIG. 3 and Tables 5A-5C below.

TABLE 5A
Skin flux data for Patch 7

| | mg Drug | | Average flux of |
|---|---|---|---|
| | Permeated/cm2 | | ketamine |
| Hours post | cumulative | | (mg/cm²*h) |
| application | Patch 7 | Interval | Patch 7 |
| 3.5 | 0.04 | 3.5 | 0.010 |
| 12 | 1.17 | 8.5 | 0.133 |
| 24 | 7.78 | 12 | 0.551 |
| 48 | 24.49 | 24 | 0.696 |
| 72 | 32.35 | 24 | 0.328 |

TABLE 5B
Skin flux data for Patch 6

| | mg Drug | | Average flux of |
|---|---|---|---|
| | Permeated/cm2 | | ketamine |
| | cumulative | | (mg/cm²*h) |
| Time, h | Patch 6 | Interval | Patch 6 |
| 2 | 0.00 | 2 | 0.00181 |
| 4 | 0.01 | 2 | 0.00473 |
| 8 | 0.07 | 4 | 0.0136 |
| 12 | 0.28 | 4 | 0.0532 |
| 24 | 5.17 | 12 | 0.407 |
| 48 | 16.89 | 24 | 0.488 |
| 72 | 21.45 | 24 | 0.190 |

TABLE 5C
Average permeation rate for the 1^{st} 18 hours.

| Patch 7 (15% gel) | | | Patch 6 (10% gel) | | |
|---|---|---|---|---|---|
| | | Average | | | Average |
| | | flux of | | | flux of |
| | | ketamine | | | ketamine |
| Time, h | Interval | (mg/cm²*h) | Time, h | Interval | (mg/cm²*h) |
| 3.500 | 3.500 | 0.010 | 2 | 2 | 0.00181 |
| 12.000 | 8.500 | 0.133 | 4 | 2 | 0.00473 |
| 18.000 | 6.000 | 0.551 | 8 | 4 | 0.01362 |
| | | | 12 | 4 | 0.05316 |
| | | | 18 | 6 | 0.40733 |

For comparison, also shown in FIG. 3 is the skin flux data of Patch 5, which is an adhesive matrix formulation prepared according to the process described in International Application No. PCT/US2016/039601, published as WO2017/003935. Skin flux data from adhesive matrix formulation, Patch 5 (DIA, drug-in-adhesive), are also presented in Table 5D below for comparison.

TABLE 5D
Comparison of skin flux data, DIR patches vs DIA patch

| Patch ID | DIR - Patch 6 | DIR - Patch 7 | DIA - Patch 5 |
|---|---|---|---|
| Patch Type | Reservoir | Reservoir | Matrix |
| Drug Loading, % | 10 | 15 | 15 |
| 1st 24 h Permeation, | 5.17 | 7.78 | 0.73 |
| mg/cm² | | | |

Results from this study: reservoir-type patch formulation (Patch 7) delivers at least 2 mg/cm²/day of ketamine and met the target delivery rate.

### Example 4. Pharmacokinetics Simulation

Convolution analysis was applied to estimate the pharmacokinetics of ketamine resulting from application of DIR and DIA patches as described above. The pharmacokinetic parameters of ketamine were based on the report from Fanta, et al., Eur. J. Clin. Pharmacol., 71:441-447 (2015). The convolution analysis for the Example patches (DIA Patch 5 and DIR Patch 6) at 200 cm² showed that the ketamine concentrations from the two patches rise slowly and plateau at round 50 ng/mL and 1000 ng/mL, respectively (FIG. 4F). Such a wide coverage of ketamine concentrations from the current DIR and DIA inventions, is able to provide sufficiently high and long-lasting exposure of ketamine for the treatment of different indications, which include depression, anxiety, pains, etc.

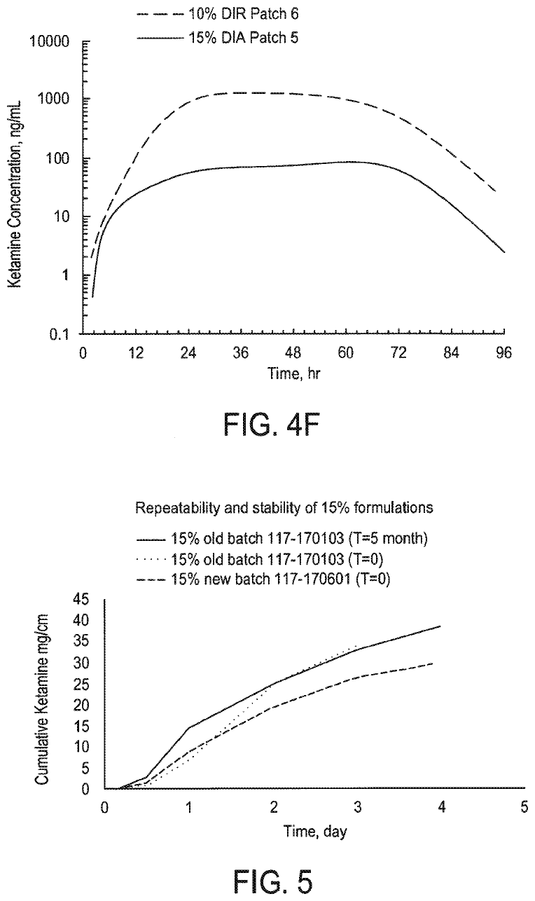

Also, based on the convolution analysis above, by modifying the amount of ketamine and/or patch size, the DIR patch can be used to generate a wide variety of pharmacokinetic profiles of ketamine. Examples of such possible PK profiles are briefly summarized in Tables 6A-6E, which are based on as those of patch 7, if configured as (or scaled to) a 72-hour delivery patch. See also FIGS. 5A-5E.

TABLE 6A
Estimated Time-Course of Plasma Ketamine Concentration
of DIR Patch Designed for 12-hour Delivery

| Time | Patch size | | | | | |
|---|---|---|---|---|---|---|
| Interval | 200 cm2 | 100 cm2 | 50 cm2 | 25 cm2 | 10 cm2 | 5 cm2 |
| (hours) | Estimated Plasma Ketamine Concentration (ng/mL) | | | | | |
| 3.5 | 16.1 | 8.07 | 4.04 | 2.02 | 0.807 | 0.404 |
| 12 | 275 | 138 | 68.8 | 34.4 | 13.8 | 6.88 |
| 24 | 22.7 | 11.4 | 5.68 | 2.84 | 1.14 | 0.568 |
| 48 | 2.56 | 1.28 | 0.641 | 0.321 | 0.128 | 0.0641 |
| 72 | 0.302 | 0.151 | 0.0754 | 0.0377 | 0.0151 | 0.00754 |
| 96 | 0.0358 | 0.0179 | 0.00895 | 0.00448 | 0.00179 | 0.000900 |

TABLE 6B
Estimated Time-Course of Plasma Ketamine Concentration
of DIR Patch Designed for 18-hour Delivery

| Time | Patch size | | | | | |
|---|---|---|---|---|---|---|
| Interval | 200 cm2 | 100 cm2 | 50 cm2 | 25 cm2 | 10 cm2 | 5 cm2 |
| (hours) | Estimated Plasma Ketamine Concentration (ng/mL) | | | | | |
| 3.5 | 16.1 | 8.07 | 4.04 | 2.02 | 0.807 | 0.404 |
| 12 | 275 | 138 | 68.8 | 34.4 | 13.8 | 6.88 |
| 18 | 1090 | 55 | 272 | 136 | 54.5 | 27.2 |
| 24 | 194 | 97.0 | 48.5 | 24.2 | 9.70 | 4.85 |
| 48 | 16.3 | 8.16 | 4.08 | 2.04 | 0.820 | 0.410 |
| 72 | 1.95 | 0.980 | 0.490 | 0.240 | 0.100 | 0.0500 |
| 96 | 0.229 | 0.115 | 0.0570 | 0.0290 | 0.0110 | 0.00600 |

TABLE 6C
Estimated Time-Course of Plasma Ketamine Concentration
of DIR Patch Designed for 24-hour Delivery

| Time | Patch size | | | | | |
|---|---|---|---|---|---|---|
| Interval | 200 cm2 | 100 cm2 | 50 cm2 | 25 cm2 | 10 cm2 | 5 cm2 |
| (hours) | Estimated Plasma Ketamine Concentration (ng/mL) | | | | | |
| 3.5 | 16.1 | 8.07 | 4.04 | 2.02 | 0.807 | 0.404 |
| 12 | 275 | 138 | 68.8 | 34.4 | 13.8 | 6.88 |
| 24 | 1233 | 616 | 30 | 154 | 61.6 | 30.8 |
| 48 | 39.7 | 19.9 | 9.93 | 4.97 | 1.99 | 0.990 |
| 72 | 4.72 | 2.36 | 1.18 | 0.590 | 0.240 | 0.120 |
| 96 | 0.560 | 0.280 | 0.140 | 0.0700 | 0.0300 | 0.0100 |

TABLE 6D
Estimated Time-Course of Plasma Ketamine Concentration
of DIR Patch Designed for 48-hour Delivery

| Time | Patch size | | | | | |
|---|---|---|---|---|---|---|
| Interval | 200 cm2 | 100 cm2 | 50 cm2 | 25 cm2 | 10 cm2 | 5 cm2 |
| (hours) | Estimated Plasma Ketamine Concentration (ng/mL) | | | | | |
| 3.5 | 16.1 | 8.07 | 4.04 | 2.02 | 0.807 | 0.404 |
| 12 | 275 | 138 | 68.8 | 34.4 | 13.8 | 6.88 |
| 24 | 1233 | 616 | 30 | 154 | 61.6 | 30.8 |
| 48 | 1712 | 856 | 428 | 214 | 85.6 | 42.8 |
| 72 | 67.7 | 33.9 | 16.9 | 8.46 | 3.39 | 1.69 |
| 96 | 8.06 | 4.03 | 2.01 | 1.01 | 0.400 | 0.200 |

TABLE 6E
Estimated Time-Course of Plasma Ketamine Concentration
of DIR Patch Designed for 72-hour Delivery

| Time | Patch size | | | | | |
|---|---|---|---|---|---|---|
| Interval | 200 cm2 | 100 cm2 | 50 cm2 | 25 cm2 | 10 cm2 | 5 cm2 |
| (hours) | Estimated Plasma Ketamine Concentration (ng/mL) | | | | | |
| 3.5 | 16.1 | 8.07 | 4.04 | 2.02 | 0.807 | 0.404 |
| 12 | 275 | 138 | 68.8 | 34.4 | 13.8 | 6.88 |
| 24 | 1233 | 616 | 30 | 154 | 61.6 | 30.8 |
| 48 | 1712 | 856 | 428 | 214 | 85.6 | 42.8 |
| 72 | 856 | 428 | 214 | 107 | 42.8 | 21.4 |
| 96 | 38.0 | 19.0 | 9.49 | 4.74 | 1.90 | 0.950 |

### Example 5. Stability and Repeatability Studies

To establish storage stability, the skin flux data was obtained for Patch 7 (batch 117-170103) at two time points: (1) about 1 week from preparation; and (2) after storage at 25° C., 60% relative humidity, for about 5 months. Visual inspection indicates no appreciable crystal formation at either of the two time points. Further, to compare skin flux characteristics from different batches of patches, a new hatch of 15% gel patches (batch 117-170601) was prepared and tested for skin flux characteristics. The new 15% gel patch was prepared according to the formulations and processes as described in Examples 1 and 2.

The skin flux data of Patch 7 (new and old) as well as the 15% gel patch from a new batch was shown in FIG. 5 and Table 7 below.

TABLE 7
Stability and Repeatability of Ketamine DIR Patches

Mean Cumulative Ketamine permeated, mg/cm2

| | 117-170103, | | 117-170103, |
|---|---|---|---|
| | 15% (after | 117-170601, | Tested after |
| | 5 months storage) | 15% (New | about 1 week |
| Day | (Patch 7) | batch) | from preparation |
| 0.1667 | 0.112 | 0.0460 | 0.0380 |
| 0.5 | 2.86 | 1.43 | 0.626 |
| 1 | 14.4 | 8.84 | 6.88 |
| 2 | 24.7 | 19.2 | 24.9 |
| 3 | 32.9 | 26.3 | 33.9 |
| 4 | 38.4 | 29.5 | |

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1-39. (canceled)

40. A transdermal delivery device comprising:
a backing layer,
a reservoir layer comprising a gel formulation comprising (1) ketamine in an amount of about 2% to about 30% by weight, (2) a solvent in an amount of about 40% to about 75% by weight, (3) a permeation enhancer of about 5% to about 25% by weight, and (4) a gel-forming agent in a gel-forming amount, wherein the solvent comprises ethanol and propylene glycol, and the permeation enhancer comprises levulinic acid and oleic alcohol and
an adhesive layer defining an active surface area,
wherein the transdermal delivery device provides one or more of the following skin flux characteristics when tested in vitro using human cadaver skin:
(a) a cumulative ketamine permeated of about 0.04 mg/cm² to about 3 mg/cm² at 12 hours post administration based on the active surface area;
(b) a cumulative ketamine permeated of about 0.8 mg/cm² to about 20 mg/cm² at 24 hours post administration based on the active surface area;
(c) a cumulative ketamine permeated of about 2.5 mg/cm² to about 65 mg/cm² at 48 hours post administration based on the active surface area;
(d) a cumulative ketamine permeated of about 3 mg/cm² to about 85 mg/cm² at 72 hours post administration based on the active surface area;
(e) an average flux of ketamine of about 0.005 mg/cm²*h to about 0.4 mg/cm²*h from 4 hours to 12 hours post administration;
(f) an average flux of ketamine of about 0.06 mg/cm²*h to about 1.4 mg/cm²*h from 12 hours to 18 hours post administration;
(g) an average flux of ketamine of about 0.06 mg/cm²*h to about 1.4 mg/cm²*h from 12 hours to 24 hours post administration;
(h) a steady state flux of ketamine of about 0.06 mg/cm²*h to about 1.8 mg/cm²*h;
(i) an average flux of ketamine of about 0.08 mg/cm²*h to about 1.8 mg/cm²*h from 24 hours to 48 hours post administration; and
(j) an average flux of ketamine of about 0.03 mg/cm²*h to about 0.9 mg/cm²*h from 48 hours to 72 hours post administration.

41. The transdermal delivery device of claim 40, wherein the adhesive layer comprises a pressure sensitive adhesive.

42. The transdermal delivery device of claim 40, further comprising an abuse deterrent agent selected from capsaicin, apomorphine, denatonium, sodium laurel sulfate, niacin and combinations thereof.

43. The transdermal delivery device of claim 40, wherein the reservoir layer has a coat weight of about 0.15 g/cm² to about 0.24 g/cm² active surface area.

44. The transdermal delivery device of claim 40, wherein the reservoir layer comprises an amount of ketamine sufficient to provide about 0.1 mg/day/cm² to about 30 mg/day/cm² of ketamine over a period of time selected from about 8 hours, about 12 hours, about 18 hours, about 24 hours, about 2 days, about 3 days, about 4 days, about 5 days, about 6 days, and about 7 days.

45. The transdermal delivery device of claim 40, wherein the reservoir layer comprises the gel formulation comprising ketamine as the only drug in an amount of about 10% by weight, ethanol in an amount of about 67% by weight, oleic alcohol in an amount of about 5.0% by weight, levulinic acid in an amount of about 7% by weight, propylene glycol in an amount of about 9.0% by weight, and hydroxypropylcellulose in an amount of about 2.0% by weight.

46. The transdermal delivery device of claim 40, wherein the reservoir layer comprises the gel formulation comprising ketamine as the only drug in an amount of about 15% by weight, ethanol in an amount of about 60.0% by weight, oleic alcohol in an amount of about 5.0% by weight, levulinic acid in an amount of about 9.0% by weight, propylene glycol in an amount of about 9.0% by weight, and hydroxypropylcellulose in an amount of about 2.0% by weight.

47. A ketamine gel formulation comprising ketamine in the amount of about 2% to about 30% by weight, a solvent in the amount of about 40% to about 75% by weight, a permeation enhancer of about 5% to about 25% by weight, and a gel-forming agent in a gel-forming amount, wherein the solvent comprises ethanol and propylene glycol, and the permeation enhancer comprises levulinic acid and oleic alcohol.

48. The ketamine gel of claim 47, wherein the permeation enhancer comprises (a) levulinic acid in the amount of about 0.1% to about 15% by weight of the gel formulation; and (b) oleic alcohol in the amount of about 0.1% to about 10% by weight of the gel formulation.

49. A transdermal delivery device comprising:
a backing layer,
a reservoir layer comprising the ketamine gel of claim 47,
an adhesive layer, and
a release liner.

50. The transdermal delivery device of claim 49, which is storage stable at room temperature.

51. The transdermal delivery device of claim 49, which is free of a crystallization inhibitor selected from polyvinyl pyrrolidone-co-vinyl acetate and polymethacrylate.

52. The ketamine gel of claim 47, wherein the permeation enhancer comprises levulinic acid in the amount of about 5% to about 15% by weight of the gel formulation and oleic alcohol in the amount of about 1% to about 8% by weight of the gel formulation.

53. The ketamine gel of claim 47, comprising ketamine as the only drug in an amount of 5-15% by weight, ethanol in an amount of 50-65% by weight, oleic alcohol in an amount of 1-8% by weight, levulinic acid in an amount of 5-15% by weight, and propylene glycol in an amount of 5-15% by weight.

54. The ketamine gel of claim 47, comprising ketamine as the only drug in an amount of about 10% by weight, ethanol in an amount of about 67% by weight, oleic alcohol in an amount of about 5.0% by weight, levulinic acid in an amount of about 7% by weight, propylene glycol in an amount of about 9.0% by weight, and hydroxypropylcellulose in an amount of about 2.0% by weight.

55. The ketamine gel of claim 47, comprising ketamine as the only drug in an amount of about 15% by weight, ethanol in an amount of about 60.0% by weight, oleic alcohol in an amount of about 5.0% by weight, levulinic acid in an amount of about 9.0% by weight, propylene glycol in an amount of about 9.0% by weight, and hydroxypropylcellulose in an amount of about 2.0% by weight.
